(19) United States
(12) Patent Application Publication
(10) Pub. No.: US 2025/0041869 A1
(43) Pub. Date: Feb. 6, 2025

(54) POINT-OF-CARE INCUBATOR FOR ISOTHERMAL NUCLEIC ACID AMPLIFICATION TESTS (PIINT) AND APPLICATIONS OF SAME

(71) Applicant: BOARD OF TRUSTEES OF THE UNIVERSITY OF ARKANSAS, Little Rock, AR (US)

(72) Inventor: Aubrey Schultz, Fayetteville, AR (US)

(21) Appl. No.: 18/792,723

(22) Filed: Aug. 2, 2024

Related U.S. Application Data
(60) Provisional application No. 63/530,725, filed on Aug. 4, 2023.

(51) Int. Cl. B01L 7/00; C12Q 1/689

(52) U.S. Cl. CPC B01L 7/52; C12Q 1/689; B01L 2300/042; B01L 2300/0609; B01L 2300/12; B01L 2300/1827; C12Q 2600/156

(57) ABSTRACT

This invention provides a point-of-care (POC) device for isothermal nucleic acid amplification tests (NAAT). The POC device comprises a housing; a heater disposed in the housing; at least one heat sink disposed in the housing in thermal communications with the heater, and configured to speed up heating and hold reaction tubes containing the NAAT being incubated; and a phase change material adapted for regulating temperature of the reactions. The POC device is a point-of-care incubator for isothermal nucleic acid amplification tests (PIINT). The POC device is used for POC detection of Johne's disease (JD) in a subject.

### CROSS-REFERENCE TO RELATED PATENT APPLICATION

This application claims priority to and the benefit of U.S. Provisional Patent Application Ser. No. 63/530,725, filed Aug. 4, 2023, which is incorporated herein in its entirety by reference.

### FIELD OF THE INVENTION

The invention relates generally to medical devices, and particularly to a point-of-care (POC) device, e.g., a point-of-care incubator for isothermal nucleic acid amplification tests (PIINT), and applications of the same.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Isothermal nucleic acid amplification tests (NAATs) are typically used to diagnose infectious diseases. Two commonly used isothermal NAATs are recombinase polymerase amplification (RPA) and loop-mediated isothermal amplification (LAMP). Isothermal NAATs reactions only progress under strict temperature ranges specific to each kind of test. RPA reactions are optimal when the temperature is in the range of 37-42° C. for between 15-30 minutes. LAMP reactions are optimal when the temperature is in the range of 60-65° C. for 30-60 minutes. The goal of the kinds of a point-of-care (POC) diagnostic devices that are in the aforementioned field is to incubate isothermal NAATs within the respective required temperature ranges for the duration of the reactions. POC tests also need to be able to be done without reliance on the expensive, sophisticated equipment found in a laboratory or clinical setting and without the need for trained professionals. Some researchers have developed POC devices for isothermal NAATs that use exothermic chemical reactions as a heat source and a phase change material (PCM) to prevent overheating. These devices are cheap and robust, but reaction components must be replaced after each test and have an inconsistent energy output dependent on manufacturing and storage conditions, as well as user expertise.

Additionally, the commonly used reactants (magnesium oxide, iron oxide, and others) can be toxic. Another common strategy for a POC device for isothermal NAATs is to use a resistive heating element and batteries controlled by a micro-controller such as an Arduino. For this method, the reaction volume must be in a microchip (as opposed to cheaper, more user-friendly reaction tubes) to maximize the surface area of the reaction in contact with the heater. In order for the micro-controller to be effective in reacting to temperature fluctuations, the large surface area is required. These devices are very good at maintaining isothermal conditions, but the micro-controller adds complexity and expense to the device, makes it harder for untrained users to repair or modify, and if anything goes wrong, the reaction is at great risk of overheating given its proximity to the reaction volume.

Therefore, a heretofore unaddressed need exists in the art to address the aforementioned deficiencies and inadequacies.

### SUMMARY OF THE INVENTION

In view of the foregoing, this invention provides a portable, inexpensive incubator capable of maintaining isothermal conditions for point-of-care (POC) isothermal nucleic acid amplification tests (NAAT). It offers a robust, minimally-instrumented, user-friendly way to diagnose infectious diseases in low-resource settings. This would allow for timely and accurate treatment for individuals in areas where diagnosing infectious diseases via traditional methods (i.e., in a clinical setting) is difficult. This would improve patient outcomes and could slow the spread of disease, benefiting both individual and global health.

In one aspect of the invention. The POC device comprises a housing; a heater disposed in the housing; at least one heat sink disposed in the housing in thermal communications with heater, and configured to speed up heating and hold reaction tubes containing the NAAT being incubated; and a phase change material (PCM) adapted for regulating temperature of the reactions.

In one embodiment, the housing is thermally insulated.

In one embodiment, the housing includes both an external insulated lid and an internal insulated plug.

In one embodiment, the heater is placed in the bottom of the housing.

In one embodiment, the heater comprises a resistive heater of a thin-film polyimide heater.

In one embodiment, the heater comprises a positive temperature coefficient (PTC) heater, a small electric or gas-powered flame, burning coal or embers, an external hot-plate or stove top, nichrome wire, and/or boiling water.

In one embodiment, the POC device further comprises a power source for operably providing power to the heater.

In one embodiment, the power source comprises batteries.

In one embodiment, the batteries are rechargeable batteries.

In one embodiment, the POC device further comprises a cup placed inside the housing in such a way that it is suspended over the heater without making direct contact.

In one embodiment, the cup is configured to hold the PCM, the reaction tubes, and the at least one heat sink.

In one embodiment, the cup is configured to hold the least one heat sink such that the at least one heat sink is in in contact with the bottom of the cup and is partially submerged in the PCM.

In one embodiment, the at least one heat sink has center holes that are configured to fill with PCM.

In one embodiment, the center holes are designed to hold the reaction tubes containing the NAAT so that the reaction volume is submerged in the PCM.

In one embodiment, the at least one heat sink is adapted to increase the thermal conductivity of the PCM allowing for rapid heating and electricity-free incubation.

In one embodiment, an interior lid is provided inside of the aluminum cup and an insulated lid is provided over the opening of the housing during incubation.

In one embodiment, the at least one heat sink is made of aluminum, copper, or the like.

In one embodiment, the cup is made of a material that is same as or different from that of the at least one heat sink.

In one embodiment, the phase change material (PCM) is adapted for regulating the temperature to avoid the complexity of a microcontroller.

In one embodiment, the PCM is adapted such that the PCM has a melting temperature close to the target reaction temperature of a given NAAT to maintain an isothermal condition.

In one embodiment, the PCM is PureTemp®-37, which has a melting temperature of 37° C., for recombinase polymerase amplification (RPA).

In one embodiment, the PCM is PureTemp®-63, which has a melting temperature of 63° C., for loop-mediated isothermal amplification (LAMP).

In one embodiment, the device has no electronic components.

In one embodiment, the POC device is a point-of-care incubator for isothermal nucleic acid amplification tests (PIINT).

In another aspect, the invention relates to a method of POC detection of Johne's disease (JD) in a subject. The method comprises preparing bio-samples of the subject; incubating the bio-samples in the above disclosed POC device; and visually detecting the JD from the incubated bio-samples.

In one embodiment, said preparing the bio-samples of the subject comprises collecting the bio-samples from the subject; cleaning and concentrating the bio-samples for DNA extraction; determining suitable primers for detecting Mycobacterium avium ssp. Paratuberculosis (MAP); and mixing the bio-samples and the primers for recombinase polymerase amplification (RPA) or loop-mediated isothermal amplification (LAMP) reactions.

In one embodiment, said incubating the bio-samples in the POC device comprises heating the POC device until the POC device reaches a target temperature; and placing the bio-samples into the center of the heat sinks and incubating them for a period of time to perform the RPA or LAMP reactions.

In one embodiment, the target temperature is about 37° C., and the period of time is about 30 minutes.

In one embodiment, the target temperature is about 63° C., and the period of time is about 60 minutes.

In one embodiment, said visually detecting the JD from the incubated bio-samples comprises mixing an amount of SYBR Green I (SG1) with the incubated bio-samples to form a mixture thereof; and illustrating the mixture with ultraviolet (UV) light at a wavelength of about 365 nm.

In one embodiment, the bio-samples that are positive for the JD fluoresce with more intensity than a negative control.

These and other aspects of the present invention will become apparent from the following description of the preferred embodiments, taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. The same reference numbers may be used throughout the drawings to refer to the same or like elements in the embodiments.

FIG. 1 shows components of the PIINT (POC Device) according to one embodiment of the invention. (A) Vacuum jar, (B) Thin-film heater inside jar, (C) Modified aluminum ramekin, (D) Ramekin containing PCM, aluminum heat sinks, and PCR tube, (E) Interior insulated lids for the ramekin, (F) Exterior insulated lid for the jar with an opening for cords.

FIG. 2 shows a PCR tube with a hole drilled for thermocouple measurements according to one embodiment of the invention.

FIG. 3 shows a configuration using a simple on/off/on switch allowing for alternating voltage between 12V and 6V. In this configuration, 8 total batteries are required, and the heater can be powered by either all 8, or only 4 of those batteries.

FIG. 4 shows a configuration for alternating voltage between 12V and 6V where two power sources with male connectors can be manually connected to the female connector on the heating element. 8 batteries are in one power source, and four are in another, requiring 12 batteries total.

FIG. 5 shows components of the PIINT (POC Device) according to one embodiment of the invention. (A) Heat sinks and PCM (PureTemp®-37) are held in an aluminum mold. This provides room for three PCR tubes. (B) A 12V polyimide thin-film heater is placed at the bottom of the thermos. (C) The aluminium mold sits on the inner lip of the thermos. (D) The thermos is covered with an insulated lid during the incubation periods. (E) The structural components of the POC device (not including the PCM)

FIG. 6 shows an experiment result using the PIINT to incubate LAMP reactions in the target temperature range (represented by the red bar) of 60-65° C. The device was connected to 12V of power in the first 30 minutes to reach the target temperature range, and then the voltage was dropped to 6V during the remaining 60-minute incubation period. The reactions reached the target temperature in 25 minutes. Because the phase change material was only partially melted at the end of testing, the reactions did not overheat. In conjunction with the data for the PIINT running RPA, this shows that the PIINT is able to reach a variety of target temperature and could incubate many isothermal reactions in a portable setting.

FIG. 7 shows a simplified graph plotting the temperature of a material as a function of energy added. This highlights the isothermal conditions present during phase change.

FIG. 8 shows an assembly of the POC device according to one embodiment of the invention. In step 1, the thin-film polyimide heater is placed in the bottom of the insulated steel vessel. In step 2, PureTemp®-37, three aluminum heat sinks, and three PCR tubes are placed in the bottom of a small aluminum cup, which is then placed inside the steel vessel suspended above the heater. In step 3, a small internal lid is placed in the internal aluminum cup. In step 4, a larger external lid is placed over the steel vessel. In step 5, before testing, the heater is connected to rechargeable batteries via a snap-connecter.

FIG. 9 shows components of the PIINT. (A) A vacuum-insulated steel vessel, (B) An aluminum cup holding PCM, aluminum heat sinks, and sample tubes, suspended in component A (B1/B2) The side (1) and top (2) view of component B, (C) An external foam insulation lid that is placed over component A, (D) An internal foam insulation lid that is placed inside component B and covers the sample tubes, (E) A 12V thin-film polyimide heater that is placed at the bottom of component A, (F) Rechargeable Ni-MH batteries used to power component E.

FIG. 10 shows the assembled PIINT device according to one embodiment of the invention.

FIG. 11 shows the temperature of the three PCR tubes during the 15-minute heating periods and the 30-minute incubation. The grey-shaded areas represent the target temperature range (37-42° C.). (A) Temperature profile from start to finish. The red line indicates the point where the power to the device was disconnected. The dashed box in (A) shows the range highlighted in (B) The average temperature of the 3 PCR tubes during incubation. Error bars represent the standard deviation across a triplicate of experiments.

FIG. 12 shows the temperatures of the three PCR tubes during a preheat and 75-minute incubation. After heating for 15 minutes, the device was disconnected from the power source, and the temperature was recorded for as long as the tubes remained in the target temperature range (≤37° C.). The grey-shaded area represents the target temperature range (37-42° C.). The red dashed line indicates the point where the power to the device was disconnected.

FIG. 13 shows temperature profiles showing the average temperature of the sets of three PCR tubes incubated consecutively in the POC device. The grey-shaded area represents the target temperature range (37-42° C.). The dashed box in (A) represents the close-up view of the temperatures seen in (B). (A) The complete temperature profile during the experiment. (B) A focused view of the temperature profile between the temperatures 37-39° C.

FIG. 14 shows the gel electrophoresis results showing the RPA product incubated in the POC device. The contents of the lanes are as follows: (1) 100 bp-4 Kb molecular ladder; (2) Positive control; (3) Negative control; (4)-(6) Samples incubated in the device.

FIG. 15 shows temperature profile of the samples inside of the PIINT during a single RPA reaction. The orange shaded area (left) indicates the time that the PIINT was connected to the power source during the initial heating stage.

FIG. 16 shows temperature profile inside the PIINT during the first 8 hours of mock RPA testing. The orange-shaded regions indicate periods when the PIINT was connected to the heater. The black diamond indicates the average temperature of the PIINT during each incubation period. The plus (+) and minus (−) symbols represent the temperature at the start and end of each testing period, respectively. Note that the y-axis of the graph has been reduced to show only the 35-40 range for clarity.

FIG. 17 shows the temperature profile inside the PIINT during the last nine incubations that were performed after the 8-hour mark. The orange-shaded regions indicate periods when the PIINT was connected to the heater. The black diamond indicates the average temperature of the PIINT during each incubation period. The plus (+) and minus (−) symbols represent the temperature at the start and end of each testing period, respectively. The temperature fluctuations between the start and end of later incubations are higher than those performed earlier. Note that the y-axis of the graph has been reduced to show only the 35-40 range for clarity.

FIG. 18 shows the temperature of the sample tubes inside the PIINT during a 30-minute warm-up phase and a 60-minute incubation phase simulating a LAMP reaction. The grey-shaded areas represent the target temperature range (60-65° C.). (A) PIINT temperature profile from start to finish. The red line indicates the point where the power to the device was switched from 12V to 6V. The dashed box shows the range highlighted in FIG. 4-2.B (B) The average temperature of the sample tubes in the PIINT during incubation. Error bars represent the standard deviation across a triplicate of experiments.

FIG. 19 shows the structure of SYBR Green I.

FIG. 20. The visual change in RPA products caused by SG1 becomes more apparent under UV light.

FIG. 21 shows RPA performed on TwistAmp® Liquid Basic positive control DNA with SG1 added. Pictures were taken in both the natural laboratory lighting (left) and under a 365/395 nm UV torch (left).

FIG. 22 shows RPA performed on MAP DNA target with SG1 added. Pictures were taken in the natural laboratory lighting (left), under a 395 nm UV torch (middle), and under a 365 nm UV torch (right).

FIG. 23 shows an example of a positive and negative RPA result using SG1 and a 365 nm UV light.

FIG. 24 shows JD RPA results containing SG1 under a 365 nm light source. The image shows the incubated product for the negative control (left), sample A (middle) and sample B (right)

FIG. 25 shows a comparison between RPA results containing SG1 incubated with different sets of MAP primers under a 365 nm light source.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present there between. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this invention, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present invention, and will not be interpreted in an idealized or overly formal sense unless expressly so defined herein.

As used herein, the phrase at least one of A, B, and C should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

This invention relates to a portable, inexpensive incubator capable of maintaining isothermal conditions for point-of-care (POC) isothermal nucleic acid amplification tests (NAAT). It offers a robust, minimally-instrumented, user-friendly way to diagnose infectious diseases in low-resource settings. This would allow for timely and accurate treatment for individuals in areas where diagnosing infectious diseases via traditional methods (i.e., in a clinical setting) is difficult. This would improve patient outcomes and could slow the spread of disease, benefiting both individual and global health.

In one aspect of the invention, the POC device comprises a housing; a heater disposed in the housing; at least one heat sink disposed in the housing in thermal communications with heater, and configured to speed up heating and hold reaction tubes containing the NAAT being incubated; and a phase change material (PCM) adapted for regulating temperature of the reactions.

In one embodiment, the housing is thermally insulated.

In one embodiment, the housing includes both an external insulated lid and an internal insulated plug.

In one embodiment, the heater is placed in the bottom of the housing.

In one embodiment, the heater comprises a resistive heater of a thin-film polyimide heater.

In one embodiment, the heater comprises a positive temperature coefficient (PTC) heater, a small electric or gas-powered flame, burning coal or embers, an external hot-plate or stove top, nichrome wire, and/or boiling water.

In one embodiment, the POC device further comprises a power source for operably providing power to the heater.

In one embodiment, the power source comprises batteries.

In one embodiment, the batteries are rechargeable batteries.

In one embodiment, the POC device further comprises a cup placed inside the housing in such a way that it is suspended over the heater without making direct contact.

In one embodiment, the cup is configured to hold the PCM, the reaction tubes, and the at least one heat sink.

In one embodiment, the cup is configured to hold the least one heat sink such that the at least one heat sink is in in contact with the bottom of the cup and is partially submerged in the PCM.

In one embodiment, the at least one heat sink has center holes that are configured to fill with PCM.

In one embodiment, the center holes are designed to hold the reaction tubes containing the NAAT so that the reaction volume is submerged in the PCM.

In one embodiment, the at least one heat sink is adapted to increase the thermal conductivity of the PCM allowing for rapid heating and electricity-free incubation.

In one embodiment, an interior lid is provided inside of the aluminum cup and an insulated lid is provided over the opening of the housing during incubation.

In one embodiment, the at least one heat sink is made of aluminum, copper, brass, or the like.

In one embodiment, the cup is made of a material that is same as or different from that of the at least one heat sink.

In one embodiment, the phase change material (PCM) is adapted for regulating the temperature to avoid the complexity of a microcontroller.

In one embodiment, the PCM is adapted such that the PCM has a melting temperature close to the target reaction temperature of a given NAAT to maintain an isothermal condition.

In one embodiment, the PCM is PureTemp®-37, which has a melting temperature of 37° C., for recombinase polymerase amplification (RPA).

In one embodiment, the PCM is PureTemp®-63, which has a melting temperature of 63° C., for loop-mediated isothermal amplification (LAMP).

In one embodiment, the device has no electronic components.

In one embodiment, the POC device is a point-of-care incubator for isothermal nucleic acid amplification tests (PIINT).

In another aspect, the invention relates to a method of POC detection of Johne's disease (JD) in a subject. The subject can be an animal, such as an infected cattle, for example, or a human.

The method comprises preparing bio-samples of the subject; incubating the bio-samples in the above disclosed POC device; and visually detecting the JD from the incubated bio-samples.

In one embodiment, said preparing the bio-samples of the subject comprises collecting the bio-samples from the subject; cleaning and concentrating the bio-samples for DNA extraction; determining suitable primers for detecting Mycobacterium avium ssp. Paratuberculosis (MAP); and mixing the bio-samples and the primers for recombinase polymerase amplification (RPA) or loop-mediated isothermal amplification (LAMP) reactions.

In one embodiment, said incubating the bio-samples in the POC device comprises heating the POC device until the POC device reaches a target temperature; and placing the bio-samples into the center of the heat sinks and incubating them for a period of time to perform the RPA or LAMP reactions.

In one embodiment, the target temperature is about 37° C., and the period of time is about 30 minutes.

In one embodiment, the target temperature is about 63° C., and the period of time is about 60 minutes.

In one embodiment, said visually detecting the JD from the incubated bio-samples comprises mixing an amount of SYBR Green I (SG1) with the incubated bio-samples to form a mixture thereof; and illustrating the mixture with ultraviolet (UV) light at a wavelength of about 365 nm.

In one embodiment, the bio-samples that are positive for the JD fluoresce with more intensity than a negative control.

Without intent to limit the scope of the invention, in the following exemplary embodiments/examples, the POC device is designed to incubate two different diagnostics tests (RPA and LAMP) at their required temperatures (37-42° C. and 60-65° C.) for the required lengths of time (30 and 60 minutes).

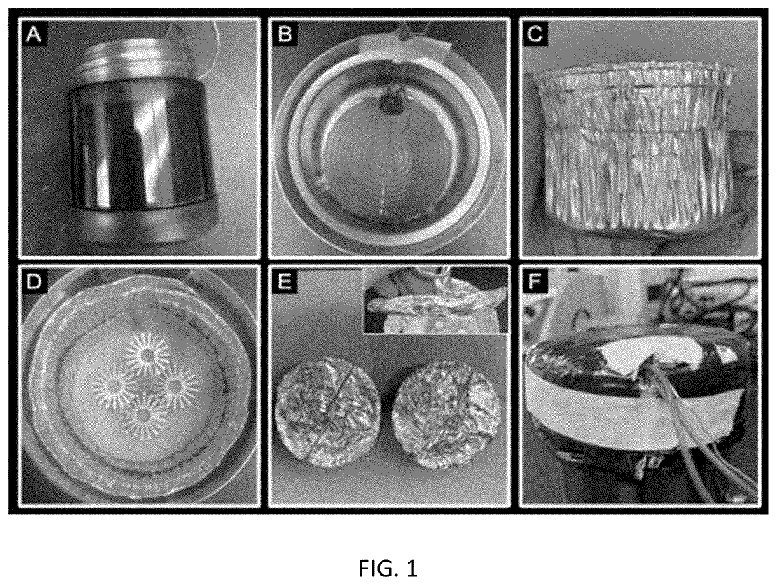

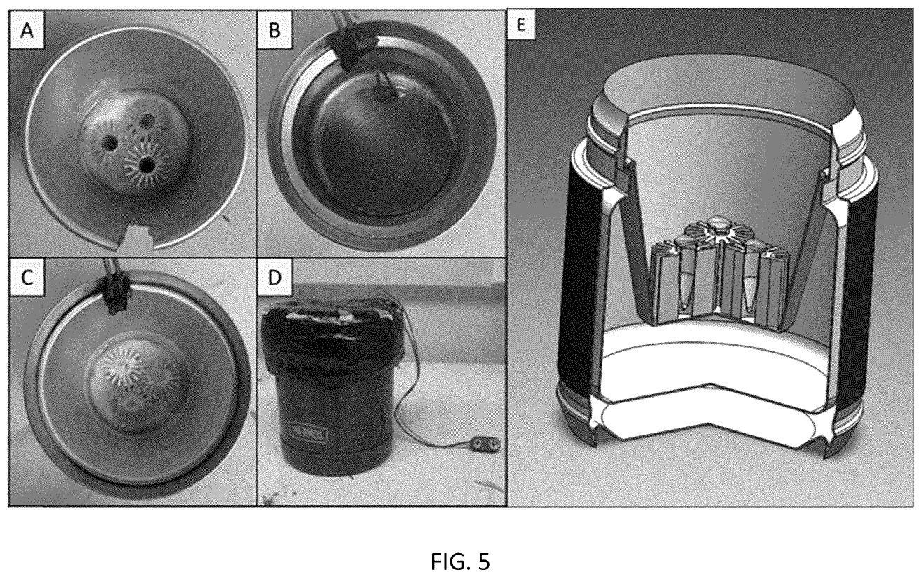

In one embodiment, the PIINT device includes an insulated vessel (e.g., a vacuum-insulated jar) with an electric heating component (e.g., a thin-film polyimide heater) at the bottom of the vessel. The device also has a smaller interior aluminum cup. The aluminum cup is used to hold aluminum heat sinks and a phase change material (PCM). The PCM that is used depends on the diagnostic test being incubated. For RPA, PureTemp®-37, which is a phase change material having a melting temperature of 37° C., is used. For LAMP, PureTemp®-63, which is a phase change material having a melting temperature of 63° C., is used. The heat sinks are in contact with the bottom of the aluminum cup are partially submerged in the PCM. The heat sinks have center holes that are allowed to fill with PCM. These center holes are about 6 mm in diameter. They are designed to hold the 0.2 mL reaction tubes containing the NAAT reaction so that the reaction volume is submerged in the PCM. The device can incubate up to 3 reactions at a time. The smaller aluminum cup is placed inside the larger insulated vessel in such a way that it is suspended over the heater without making direct contact. A small interior lid is placed inside of the aluminum cup and a large insulated lid is placed over the opening of the vessel during incubation. The heater can be powered by any source capable of reaching a voltage of 12V (e.g., solar power, the electric power grid, USB-connected power banks, etc.), but the PIINT typically is powered via rechargeable AA Ni-MH batteries. FIG. 1 shows a first prototype of the PIINT device (“PIINT Prototype 1”). FIG. 5 shows images and construction of the newest prototype of the PIINT device (“PIINT Prototype 2”).

Because of the differences in temperature and reaction length, the PIINT is operated slightly differently depending on if the NAAT being incubated is RPA or LAMP. In order for the reactions to stay in the desired temperature ranges, the PCM must absorb enough energy so that it begins to transition from a solid to liquid phase but never fully melt; if the PCM fully melts, the NAAT will overheat, stopping the reaction. Therefore, different NAATs require different conditions. The following paragraphs will describe the operating conditions for each NAAT in question.

For RPA: 20 grams of PureTemp®-37 acts as the PCM. The goal is for the reaction volumes to reach within +/−1° C. of the target temperature range (37-42° C.) within 15 minutes of the heater being connected to 12V of power. The reaction volumes must remain in the target temperature range for a minimum of 30 minutes. After the initial 15-minute warm-up phase, the device is disconnected from the power source and is entirely electricity-free. Under standard conditions, if the device is connected to the heater for 15 minutes, the incubator remains in the RPA temperature range for 75 more minutes if the lid is not opened.

For LAMP: 30 grams of PureTemp®-63 acts as the PCM. The goal is for the reaction volumes to reach within +/−2° C. of the target temperature range 60-65° C.) within 30 minutes of the heater being connected to 12V of power. The reaction volumes must remain in the target temperature range for a minimum of 60 minutes. After the initial 30-minute warm-up phase, the voltage going to the heater is dropped from 12V to 6V. Under standard conditions, if the heater is connected to a 12V power source for a 30-minute warm-up and the voltage is dropped to 6V for the incubation period, the reactions volumes stay within the LAMP temperature range for 60 minutes.

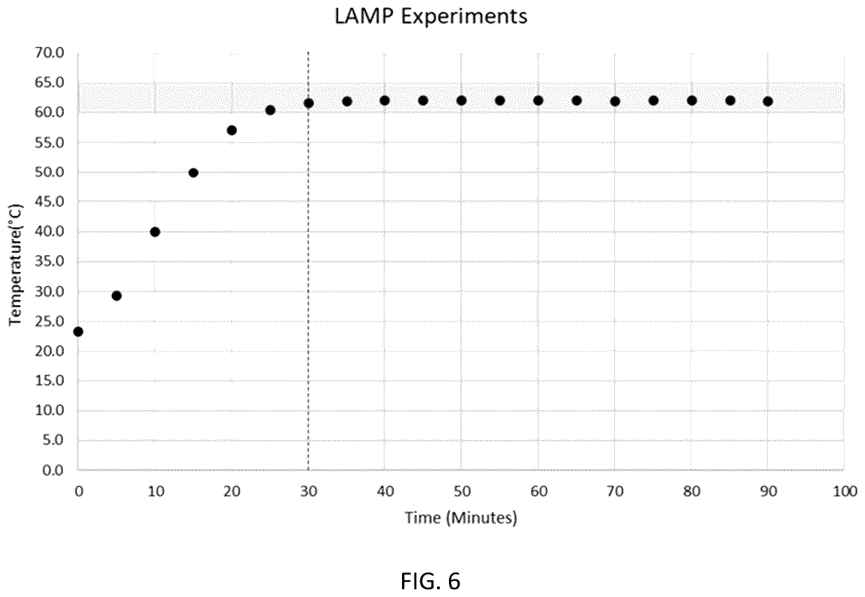

FIG. 6 shows an experiment using the PIINT to incubate LAMP reactions in the target temperature range (represented by the red bar) of 60-65° C. The device was connected to 12V of power in the first 30 minutes to reach the target temperature range, and then the voltage was dropped to 6V during the remaining 60-minute incubation period. The reactions reached the target temperature in 25 minutes. Because the phase change material was only partially melted at the end of testing, the reactions did not overheat. In conjunction with the data for the PIINT running RPA, this shows that the PIINT is able to reach a variety of target temperature and could incubate many isothermal reactions in a portable setting.

TABLE 1
Time and average temperature
in the experiment.

| | Average |
|---|---|
| Time | Temperature |
| (Minutes) | (° C.) |
| 0 | 23.3 |
| 5 | 29.2 |
| 10 | 40.0 |
| 15 | 49.9 |
| 20 | 57.1 |
| 25 | 60.5 |
| 30 | 61.6 |
| 35 | 61.9 |
| 40 | 62.1 |
| 45 | 62.1 |
| 50 | 62.1 |
| 55 | 62.1 |
| 60 | 62.0 |
| 65 | 62.0 |
| 70 | 62.0 |
| 75 | 62.0 |
| 80 | 62.0 |
| 85 | 62.0 |
| 90 | 61.9 |

In one embodiment, using mock reaction volumes (50 μL water) experimentation confirming the operating conditions described above for LAMP and RPA has been performed and documented.

In one embodiment, using mock reaction volumes to represent NAATs, 10 RPA incubation periods (30 reactions) were performed concurrently with minimal heating between incubation periods; the device remained in the target RPA temperature range for 300 minutes with only 45 cumulative minutes of heating required.

In one embodiment, live RPA reactions have been successfully performed using the PIINT as an incubator and comparing the results to RPA reactions incubated in a commercial heat block.

In one embodiment, a color-changing phase change material be used to give a visual indicator of the reaction temperature, and a lid with a clear view-port is designed. The design principles of the PIINT is applied to a smaller prototype.

There are several elements that make the PIINT advantageous and unique compared to other similar devices:
1) The combination of using a PCM with an electric heater as opposed to an exothermic chemical reaction or micro-controller to raise and regulate heat.
2) The intentional air space left between the heater and the reaction volumes. Most devices with electric heaters have a microchip or tube in direct contact with the heater. Having hot air be the medium in contact with the aluminum cup holding the reaction volume allows for uniform heating of the PCM with no fear of overheating the bottom of the reaction. Additionally, the air helps dampen the effects of ambient or local temperature fluctuations.
3) For RPA, the fact that after 15 minutes of heating, the power can be completely disconnected, and the reaction remains in the correct temperature range for an additional 75 minutes—this is 45 minutes longer than for RPA reactions.
4) The fact that power can quickly be connected and disconnected means that there is very little setup required to perform concurrent reactions, and the PCM and hot air are excellent at dampening temperature fluctuations, so the device does not lose much heat when opening it to remove finished reactions and insert new ones (this is shown in the attached manuscript and will be elaborated on below).
5) For LAMP, the fact such a high-temperature range can be maintained using a simple on/off/on switch to change between two distinct voltages rather than a micro-controller constantly monitoring the voltage makes the device extremely simple compared to others.
6) The PIINT is cheaper (by hundreds to thousands of dollars-PIINT cost around $60 dollars to construct using amazon purchases). It has a much more simple user interface (on-off is the only user input). Many of the devices labeled as “portable” still require being plugged into a central power grid. It does not have a computer system at all, so its prone to breaking. For RPA, the PIINT is not even using electricity the majority of the time. The PCR tubes are not in contact with the heater directly, so they are less likely to overheat or to heat unevenly.

Briefly, the PIINT is an equipment-free, simple, robust, and affordable way to incubate isothermal nucleic acid amplification reactions for diagnosing infectious diseases. It is capable of incubating different reactions under different isothermal conditions with minimal modification; it can incubate a RPA reaction between 37-42° C. for over 30 minutes and a LAMP between 60-65° C. for 60 minutes. The PIINT uses an electric battery-powered heater to provide energy, making it portable and user-friendly. Temperature regulation does not require a control system, meaning the device has no delicate or expensive parts, making it rugged and difficult to break, even in low-resource settings. The PIINT can hold three PCR tubes at a time, and does not require any kind of custom microchip or other equipment to hold the reaction.

According to the invention, through changing the phase change material, and in some embodiments without changing anything, the PIINT should be able to incubate more isothermal NAATs than just RPA and LAMP. These amplification methods include, but are not limited to, in Table 2:

TABLE 2
Various amplification methods

| | | Reaction | Reaction |
|---|---|---|---|
| | | Temp | length |
| Amplification method | Acronym | (° C.): | (min): |
| Cross priming amplification | CPA | 63 | 30-60 |
| Exponential amplification | EXPAR | 55-60 | 30 |
| reaction | | | |
| Helicase-dependent | HDA | 37, 65 | 60-180 |
| amplification | | | |
| Multiple Displacement | MDA | 25-35 | 60-90 |
| Amplification | | | |
| Nucleic acid sequence-based | NASBA | 41 | 90 |
| amplification | | | |
| Polymerase spiral reaction | PSR | 61-65 | 60 |
| Rolling circle amplification | RCA | 30-65 | 60 |
| Strand displacement | SDA | 37-49 | 20-120 |
| amplification | | | |
| Signal-mediated amplification | SMART | 41 | 180 |
| of RNA technology | | | |

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### EXAMPLE 1

### Point-Of-Care Incubator for Isothermal Nucleic Acid Amplification Tests (PIINT)

### Design Principles

Using simple resistive (Joule) heating with no microcontroller or programing as an energy source.

Using phase change material (PCM) as a temperature regulator.

For recombinase polymerase amplification (RPA), the goals are (1) to incubate the reaction in the temperature range (37-42° C.), (2) to reach the target temperature range w/in 15 minutes, and (3) to hold the device at the target temperature for 30 minutes.

For loop-mediated isothermal amplification (LAMP), the goals are: (1) to incubate the reaction in the temperature range (60-65° C.), (2) to reach the target temperature range w/in 30 minutes, and (3) to hold the device at the target temperature for 60 minutes.

### Device

The device includes a 12V resistive heater, rechargeable Li-MH batteries as a power source, PCM being PureTemp®-37 for RPA and PureTemp®-63 for LAMP, computer heat sinks to speed up heating, and capable of holding three PCR tubes at a time.

Total prototype device cost is about $60 that includes the $25 battery charger for the Li-MH batteries.

### Recombinase Polymerase Amplification (RPA)

It takes about 15 minutes to heat the device using a 12V resistive heater.

After 15 minutes, the device is in the target temperature range (37-42° C.) and the power is disconnected.

The reactions remain in the target temperature range for the desired reaction time (about 30 minutes) for much longer (about 75 minutes without any power).

The voltage drop in the rechargeable batteries after the 15 minutes of heating is about 0.5V (11.5V to 11.0V).

Live reactions show successful amplification.

10 concurrent incubations were performed (30 mock reactions).

The device is very resistant to temperature fluctuations as a result of opening the lid.

Only very short heating periods were required between the power-free incubation periods.

The 10 incubations were done in about 345 minutes (5.75 hours). The heater was disconnected and the device was power-free for about 300 minutes. Power was only connected 13% of the time.

The voltage drop in the rechargeable batteries after the 45 minutes of heating is about 1.5V (11.5V to 10.1V).

The device could have incubated more reactions concurrently if desired.

### Loop Mediated Isothermal Amplification (LAMP)

For LAMP, data represents experiments done with a prototype.

It takes about 30 minutes to heat the device using a 12V resistive heater.

After 60 minutes, the device is in the target temperature range (60-65° C.) and the power is dropped from 12V to 6V.

The reactions remain in the target temperature range for the desired reaction time (60 minutes).

A simple ON/OFF/ON switch can be used with 8 batteries to keep the device design simple and small.

New model is optimized for LAMP.

Color-changing PCM is used as temperature indicator to remove the need for electronic temperature monitoring.

Model device using COMSOL Multiphysics to determine the effects of ambient temperature.

A miniaturized version of the device is created.

### PIINT Prototype 1

Assembly was done inside an insulated jar (FIG. 1, A). To assemble the device, the thin-film heater was first placed in the bottom of the insulated jar (FIG. 1, B). An aluminum ramekin was modified to fit into the jar (FIG. 1, C). Four round aluminum heat sinks were placed into the aluminum ramekin, and PCM was poured on top. PureTemp®-63, which has a melting point of 63° C., is used for LAMP incubation. PureTemp®-37, which has a melting point of 37° C., is used for RPA incubation. The ramekin, heatsinks, PCM, and a 0.2 mL PCR tube were then heated on a hotplate to remove air from the PCM and to ensure that the bottom of the heatsinks was in contact with the bottom ramekin (FIG. 1, D). This step is important in guaranteeing efficient heat transfer from the heater to the PCM. To insulate the reaction, small internal lids were constructed to go inside the ramekin (FIG. 1, E). Two internal lids were used. A larger insulated lid with a hole for electric cords was made for the jar (FIG. FIG. 1, F).

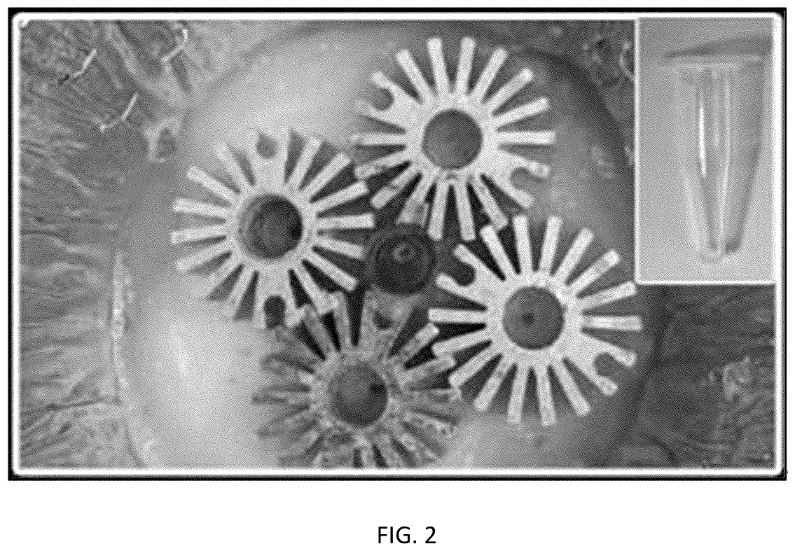

To confirm that the temperature measurement outside of the PCR tube was representative of the temperature inside the tube, an experiment was conducted that measured the temperature of both areas simultaneously. A small hole was drilled into the lid of a PCR tube containing a mock reaction volume, and a thermocouple was inserted into the lid (FIG. 2).

For RPA incubation, PureTemp®-37 is used. The heating element is initially connected to a 12V power source. As the reaction volume approaches the desired temperature range (37-42° C.), the power supply is disconnected for the remainder of the incubation period.

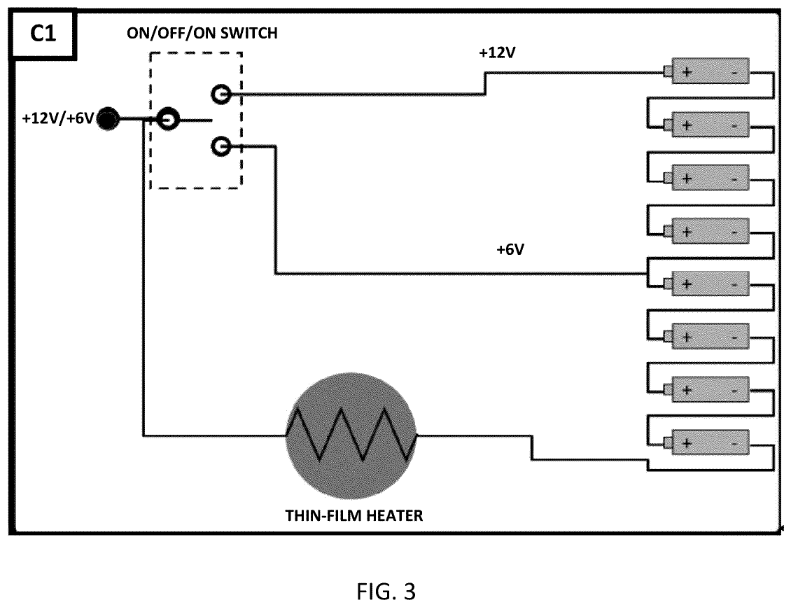

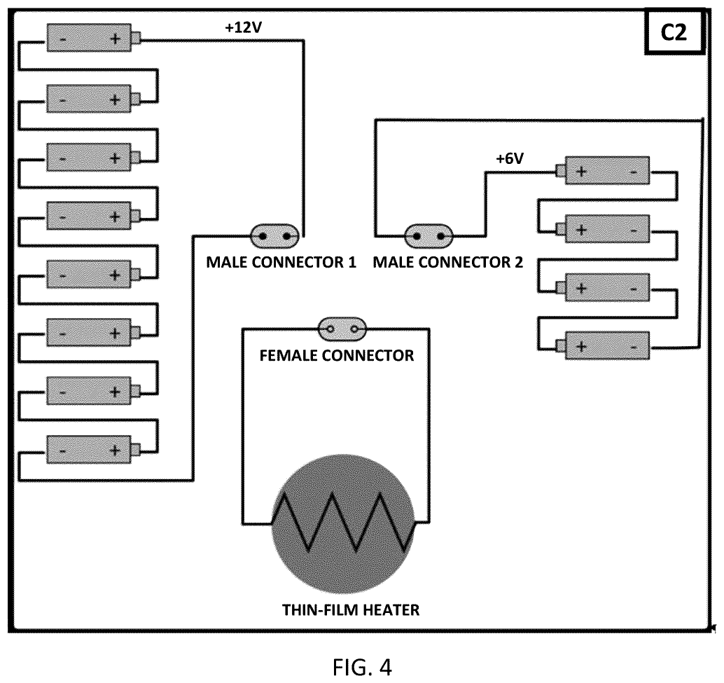

For LAMP, two voltages are used over the course of the test. The heater is initially connected to a higher voltage (12V) and then dropped to a lower voltage (6V) once the reaction volume approaches the desired temperature range (60-65° C.). After the voltage is changed from 12V to 6V, the heater remained connected to the 6V power supply for the remainder of the incubation period. Two configurations can be used to achieve this through battery power. One configuration (C1) used a switch and allowed for 8 AA batteries in total to be used; then half of the batteries are disconnected once the switch is thrown from one side to the other (FIG. 3). The second configuration (C2) used 12 batteries in total and required manually connecting and disconnecting a clip connector to change the voltage (FIG. 4).

### PIINT Prototype 2

The main components of the POC device are housed inside a 10-ounce stainless steel vacuum-insulated vessel (Thermos LLC, Schaumburg, IL, USA). This provided both housing and insulation during the reactions (see FIG. 5, D). A 12V/13W 70 mm diameter thin film polyimide heater (ICstation, Guangdong, China) was placed in the bottom of the vessel (see FIG. 5, B) A modified aluminum mold holds the PCM (PureTemp®-LLC, Plymouth, MN, USA), PCR tubes, and aluminum heat sinks (see FIG. 5, C).

PureTemp®-37 has a melting temperature of 37° C. The aluminum heat sinks allowed for increased thermal conductivity in the PCM. PCR tubes were placed inside of the center holes within the heat sinks. Using a hot plate, the PCM was melted within the mold before the heat sinks were placed (FIG. 5, A). While it was still semi-soft, PCR tubes were placed within the heat sinks to shape the PCM. The structural components of the device can be seen in FIG. 5, E. Power is provided by eight 1.2V nickel metal hydride (Ni-MH) AA batteries (EBL Mall, El Monte, CA, USA). A lid constructed from foam insulation is used during incubation (FIG. 5, D).

### EXAMPLE 2

### Detection of Johne'S Disease Using the Point-Of-Care Incubator for Isothermal Nucleic Acid Amplification Tests (PIINT)

By changing the PCM and heating conditioned in the PIINT, DNA from the bacterium that causes Johne's disease, M. avium paratuberculosis, can be incubated in the PIINT and detected using RPA and LAMP.

### Objectives

The PIINT can be used to incubate NAATs, which is demonstrated through the detection of Johne's disease. This is done by (1) optimizing the PIINT for use with LAMP, as has been done for RPA, (2) using color-changing PCM to remove the need for a thermocouple, thus reducing the instrumentation in the device, (3) using the device to detect Johne's disease via RPA, and (4) using the device to detect Johne's disease via LAMP.

### Methods

LAMP Mock Reaction Study: While the target temperature range for LAMP (60-65° C.) has been shown in an earlier prototype of the PIINT, the newest version of the PIINT has not been optimized for LAMP. To do this, PCR tubes holding mock reaction volumes (50 μL water) is used instead of live reactions. The target parameters for the heating profile are as follows:

The device must reach the target temperature range within 30 minutes.

The device must hold the mock reactions in the target temperature range for 60 minutes.

To achieve this, the device is connected to a high voltage for the warm-up period, then lowered for the incubation period; this method was successful in the earlier prototype. The previous prototype used 30 grams of PureTemp®-63 as the PCM, was connected to 12V for the warm-up period, and 6V during incubation. While optimizing the conditions for the updated PIINT, these conditions will be tested to confirm their relevancy. Then the PCM, warm-up voltage, and incubation voltage will be varied in an attempt to achieve a faster warm-up or a lower operating voltage.

Color-changing PCM Study: Because the goal is to make a device as minimally instrumented as possible, a visible indication that the device is reaching the target temperature is desired. A PCM with the same melting temperature that changes color as it melts is used instead of PureTemp®-37. Changes to the internal and external lid are modified to make a viewing port, and a small battery-powered LED light is installed inside the PIINT. Initially, a thermocouple is used to record the temperature alongside photographs of the PCM; this is done to see how accurate the color change is as an indicator of the target temperature range. Next, this method is used to incubate RPA reactions to confirm whether the color-changing PCM can be reliably used.

RPA/LAMP Johne's Disease Study: After the device is fully optimized, RPA and LAMP are used to detect Johne's disease using the PIINT. Primers and the target DNA sequence are designed based on existing literature.

Reactions incubated in the PIINT are compared to reactions incubated in a heat block. Gel electrophoresis will be used for detection. If the detection of Johne's disease is unsuccessful, an alternative livestock disease is identified.

### EXAMPLE 3

### Evaluation of New Geometries for POC Device

Using a PCM and a resistive heater as an incubator for isothermal NAATs is a versatile concept that can be used as a framework to make a variety of devices in different geometries.

A PCM and resistive heating element can be used to make devices having a variety of geometries and conditions, but through simulation and prototypes, all are able to meet the target temperature ranges for LAMP (60-65° C.) and RPA (37-42°).

### Objectives

Using methods developed first in the PIINT, multiple new geometries for POC devices can be developed. This is accomplished by (1) using the software COMSOL Multiphysics to create simulations of various prototypes under POC conditions, (2) building prototypes both informed by and independent of the COMSOL simulations, and (3) using data from prototypes to first validate COMSOL models, and then using COMSOL models to evaluate the use of the POC devices under hot and cold weather conditions. While the studies in this project can all be performed individually, the results from one also validate and inform others.

### Methods

COMSOL Multiphysics Studies: COMSOL Multiphysics is used to model a variety of device geometries. Two definite geometries are sure to be modeled, with room in the timeline granted for exploring additional geometries. The two certain models are:
1) A microchip to hold the reaction volumes rather than a PCR tube.
2) A miniaturized version of the PIINT capable of incubation a single PCR tube rather than three.

Both geometries should, in theory, use less energy and be more portable, although they might also be more delicate and, in the case of the microchip, need more specialized parts. The trade-offs are considered acceptable because different POC conditions might necessitate that different areas of the ASSURED guidelines take more president than others. Time-dependent COMSOL studies will be developed first under ideal conditions and then under more realistic POC conditions.

Prototype Study: The miniaturized PIINT device is constructed as a prototype. The vessel is no larger than 1.5 in³. It undergoes all the testing and optimizing that the PIINT went through; this includes varying the amount of PCM, voltage, and warm-up times used for NAAT incubation and creating heating profiles for mock reactions under these conditions. Ultimately, after optimizing the device, the goal is to use the device to incubate live reactions for RPA and LAMP. Should another promising prototype be designed using COMSOL, this may also undergo the study described above. This possibility is considered in the project timeline.

Prototype Validation & Weather Studies: During POC testing, climate control is not guaranteed in the testing area. For this reason, devices should be capable of operating in various situations. After building physical prototypes, COMSOL models are developed, and the data from the prototypes are used to validate the COMSOL model. After the results from the model and the physical prototype agree under standard conditions, the COMSOL models are used to investigate the behavior of the devices under other conditions. The temperature profiles of the devices under hot (32-37° C.) and cold (0-10° C.) ambient conditions are determined. This shows the flexibility of the devices and if changes need to be made based on weather conditions.

### EXAMPLE 4

### A Simple POC Device for Temperature Control of Multiple Reactions During Recombinase Polymerase Amplification (RPA)

For individuals with infectious diseases, early and accurate diagnosis is critical. A rapid diagnosis allows for prompt and effective treatment and increases the chance of a full recovery without complications. Additionally, when containing a wide-scale infectious disease outbreak, circumstances are significantly improved by the ability to test the populace frequently, swiftly, and affordably. Regarding specificity and sensitivity, nucleic acid amplification tests (NAAT) are one of the best options for diagnosing infectious diseases. Historically, polymerase chain reaction (PCR) has been used, but complex thermocycling and complicated PCR protocols have often limited PCR to clinical settings. Due to increased simplicity, the isothermal NAAT recombinase polymerase amplification (RPA) has the potential to deliver reliable POC diagnostics in low-resource settings. When designing POC devices for isothermal NAATs, creating isothermal temperature conditions is perhaps the most significant challenge. This exemplary work presents a flexible and robust device capable of incubating 3 RPA reactions for simultaneous amplification in conditions conducive to POC testing. The device costs about $60 USD to construct and is easy to assemble. A battery-powered polyimide thin-film resistive heater provides energy, and the device only requires power for a fraction of the total incubation time. The device uses a phase change material (PCM) to regulate temperature to avoid the complexity of a microcontroller. RPA reactions were successfully incubated in 30 minutes using the device.

### Introduction

Infectious diseases kill millions of people across the world each year. Infectious diseases are communicable diseases caused by pathogens such as viruses, bacteria, parasites, or fungi. It is estimated that up to half of the world population is at risk for infectious diseases; the World Health Organization (WHO) found that three of the top ten causes of death involve infectious diseases. It is clear from these statistics that infectious diseases can be highly damaging on an individual level. However, they are also uniquely damaging on a communal and societal level. Because infectious diseases can be transmitted from person to person, they spread exponentially through a community in a very short amount of time. This poses a significant risk to overall public health. This reality has never been clearer than in the wake of the SARS-CoV-2 pandemic, when, at the time of writing, the WHO reports that there have been 561,156,416 confirmed cases and 6,365,510 deaths as a result of the disease.

The risk of death from infectious disease is higher in developing countries where testing must be done in low-resource settings. The availability of testing is considered a significant variable in slowing the spread of disease and improving patient outcomes. As a result, POC diagnostics are vital for increasing the volume of testing performed worldwide and, therefore, increasing individual and global health as a whole. POC tests provide on-site results without the need to transport specimens to a laboratory. According to the WHO, a shocking 70-90% of medical devices donated to developing countries never work as intended. This highlights the need for robust and simple devices. The WHO has created a list of criteria for evaluating POC devices, the “ASSURED” standards. These criteria and how they relate to POC devices are detailed in Table 3.

Since the development of the PCR in 1983, NAATs have been a benchmark tool for diagnosing infectious diseases. PCR, while very selective and specific, has drawbacks as a POC method. PCR requires energy-extensive thermal cycling performed by trained professionals in laboratory settings with sophisticated equipment. These conditions keep PCR from being “User-friendly,” “Equipment-free,” and “Deliverable.” One of the big challenges of developing POC NAATs is temperature control. The development of isothermal NAATs has made temperature control in POC devices significantly more manageable. One isothermal NAAT, RPA is a very promising method.

TABLE 3
WHO ASSURED specifications

| Criteria | Interpretation |
|---|---|
| Affordable | Test cost <10 USD; |
| | Device cost <500 USD |
| Sensitive | Few false negatives |
| Specific | Few false positives |
| User-friendly | Simple and requires little training |
| Rapid and Robust | Enables same-day treatment; Does not |
| | require refrigerated storage |
| Equipment-free | Compact, non-electric, or battery- |
| | powered; No lab equipment |
| Deliverable | Portable, hand-held |

RPA is a NAAT that requires minimal sample preparation, can utilize freeze-dried reagents and has an optimal operating temperature range of 37-42° C. This operating range is low in contrast to the high temperatures required by PCR (95° C.) or even similar isothermal NAATs such as loop-mediated isothermal amplification (60-65° C.). RPA was first developed by Piepenburg et al. in 2006. The NAAT has since been commercialized by the biotech company TwixtDx. RPA can amplify target DNA to detectable levels in as little as 15-30 minutes. As a result, RPA is well suited for use as a POC test. Despite the favorable elements, temperature control is still a significant concern. While RPA will run at room temperature, it takes much longer for the reaction to finish under those conditions. Usually, RPA is performed using a heat block or water bath during the incubation stage. These methods are too energy extensive and expensive for a POC setting. While RPA has one of the lowest operating temperatures out of NAATs, maintaining a tight range of 37-42° C. in a device following ASSURED guidelines poses a challenge. Researchers have found ways to manage the temperature control for isothermal NAATs in a wide variety of ways. However, two methods, chemical and electrical heating, are the most prevalent. Both methods have their own benefits and downsides.

Chemical heating typically involves an exothermic reaction provided heat that is regulated via a phase-change-material (PCM). Chemical heating is often used in devices that must be robust, simple, and disposable. Two of the most common reactions used are Mg—Fe alloys and water, which is the reaction in Meal-Ready-to-Eat (MRE) military ration heaters, and Iron-Oxide reactions, which are used in hand warmers. In these devices, using a PCM prevents the reactions from overheating; picking a PCM with a melting temperature close to the target reaction temperature of a given NAAT can maintain isothermal conditions. These devices are unique in that they have no electronic components. While this can be a strong positive, the reactants must be replaced after every use, and users must be trained on how to start the reactions.

In these devices, the temperature is usually regulated using a phase change material although creative exceptions exist. PCM prevents the reactions from overheating by absorbing excess heat generated. Choosing a PCM with a melting temperature close to the target reaction temperature of a given NAAT can provide an isothermal environment for the reactions. This is done by taking advantage of thermodynamic properties that occur when a material is in its latent form during phase change. Equation (1) shows the different factors that govern total energy absorption in the stages before, during, and after a solid-to-liquid phase change.

$$Q = {mc}_{p , solid} ( {T}_{m} - {T}_{initial} ) + m Δ {H}_{f} + {mc}_{p , liquid} ( {T}_{final} - {T}_{m} ) \qquad ( 1 )$$

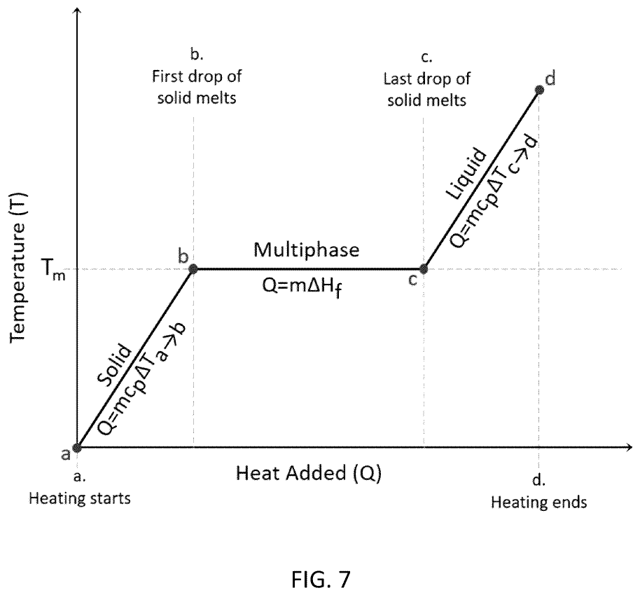

In this equation, Q represents total heat absorbed, m represents the mass of the PCM, Cₚ represents heat capacity at constant pressure, ΔHf represents the latent heat of fusion, and Tₘ represents the melting point temperature of the PCM. During phase change, as soon as the PCM reaches Tₘ and enters a multiphase state, the entire bulk of the material remains at the Tₘ until enough energy is absorbed to overcome the latent heat of fusion and fully transition to the new phase. This concept can be visualized by graphing temperature versus heat absorbed, as shown in FIG. 7. By adding enough energy to a PCM to begin the transition but not enough to overcome the heat of fusion, the material can be kept in an isothermal state at the melting point. This makes the choice of PCM very important in designing NAAT POC devices.

For the temperature ranges typical in isothermal NAATs, suitable PCMs usually are comprised of organic compounds such as fatty acids, alkanes, and paraffin waxes. In addition to the favorable temperature ranges, these materials are non-toxic and biodegradable, which makes them ideal for disposable devices. However, these PCMs typically have low thermal conductivities, necessitating the addition of thermally conductive materials such as graphene nanoparticles, aluminum wool, or aluminum heat sinks. At the time of writing, significantly more research has been published focused on using chemical heating and PCMs for POC devices designed for LAMP compared to those focused on RPA. Because LAMP has a relatively high reaction temperature (60-65° C./140-149°F.), common reactants such as those mentioned previously can be used without overheating. For RPA, however, fewer studies exist, and those that do use paraffin wax with a melting temperature of 44° C./111.2° F. as a PCM and utilize very mild exothermic reactions to prevent overheating. Ideally, a PCM with a melting point closer to 37° C. would allow for faster and more efficient heating. However, designing an exothermic reaction to heat a device quickly but not overheat the PCM can be difficult, especially at these lower temperatures. This is one area where electric heating may have advantages.

Electrical heating generally uses a resistive heating element regulated by a control system such as a microcontroller. Resistive heating, also called Joule heating, occurs when electricity passes through an electrically resistive material. Equation (2) shows the formula for Joule heating where V represents voltage, t represents time, and R represents resistance.

$$Q = {V}^{2} t / R \qquad ( 2 )$$

The energy generated is very predictable when controlling the voltage provided and the resistive element used. Some examples of suitable elements include thin-film heaters, printed circuit boards, thermistors, electrical resistance tape, and nichrome wire. For POC devices, the voltage is usually provided via batteries and regulated via a microcontroller. The electrical controls are more user-friendly than chemical heating, and using a microcontroller can allow for temperature fluctuations in the ambient environment to be accounted for during testing. However, using a microcontroller adds high cost and complexity to a device. Additionally, these devices typically require a microchip because of the need for rapid feedback in the temperature control loop. Microchips are not as accessible and cost-effective as, for example, PCR tubes, which are most used in NAATs.

The device presented in this work combines elements of both types of temperature control to create a robust, simple, inexpensive, and very user-friendly POC device. The device uses a PCM and thermos housing, and a polyimide thin-film heater powered by rechargeable batteries. On the most superficial level, the device works by maintaining the balance shown in Equation (3).

$${mc}_{p , solid} ( {T}_{m} - {T}_{initial} ) < ( {V}^{2} t / R ) < m Δ {H}_{f} \qquad ( 3 )$$

Innovation comes from the low-energy method of heating and the use of heat sinks to increase the thermal conductivity of the PCM, allowing for rapid heating and electricity-free incubation. The resulting device can incubate three RPA reactions consecutively in a manner that is very compliant with WHO ASSURED guidelines.

### Materials and Methods

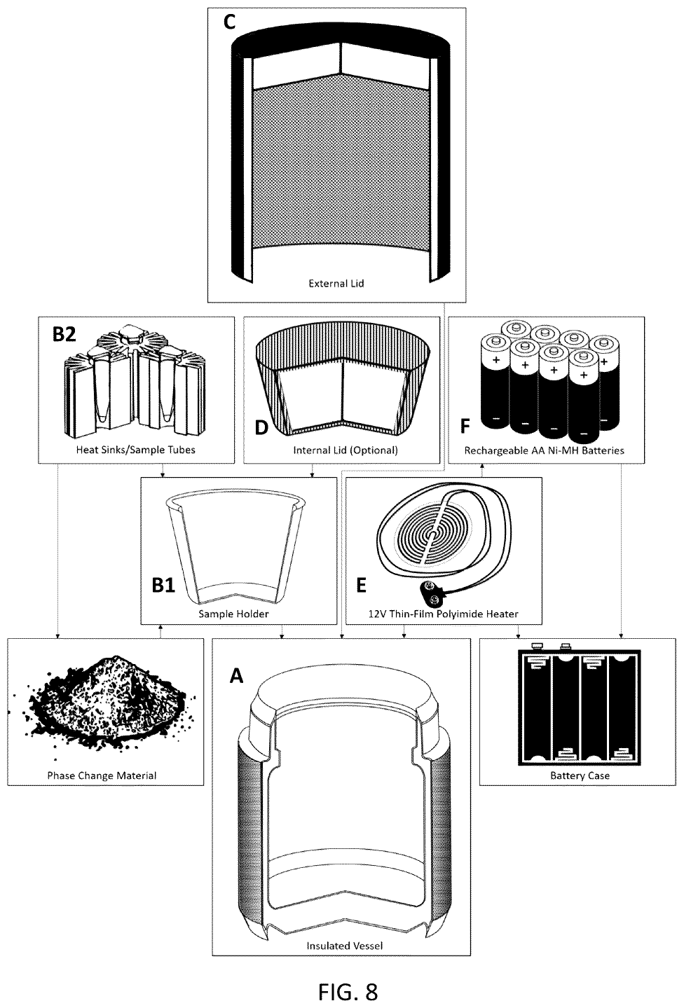

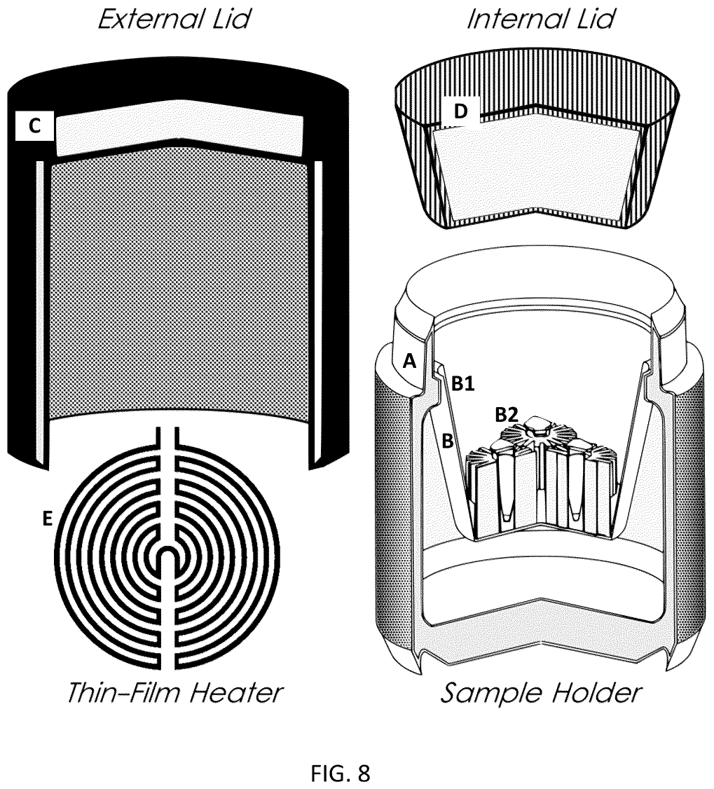

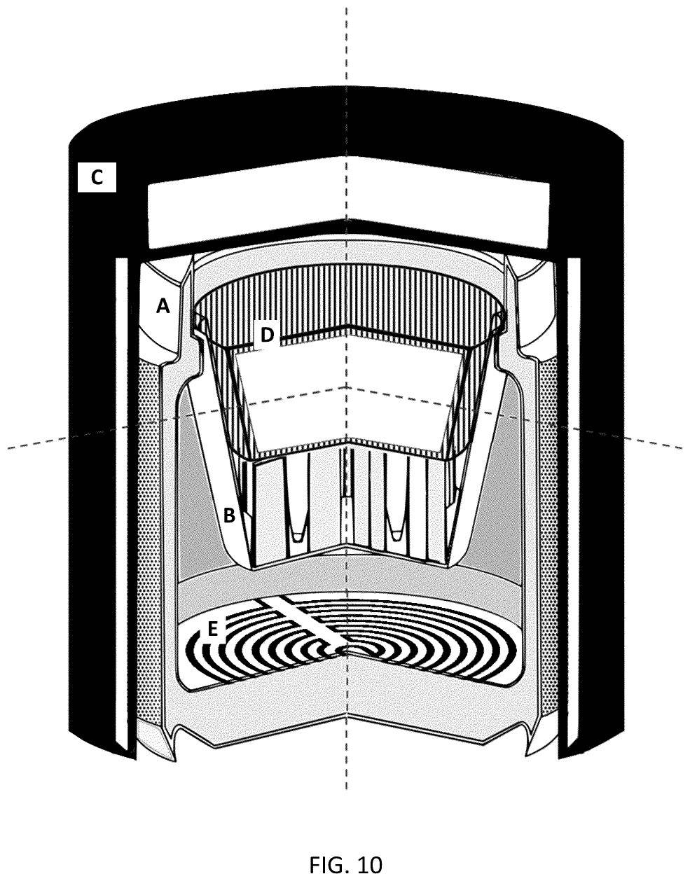

Device Assembly: The main components of the POC device are housed inside a 10-ounce stainless steel vacuum-insulated jar (Thermos LLC, Schaumburg, IL, USA). This provided both housing and insulation during the reactions. A 12V/13 W 70 mm (2.76 in.) diameter thin film polyimide heater (ICstation, Guangdong, China) was placed in the bottom of the jar. A modified aluminum baking mold holds 20 grams of the PCM (PureTemp®-37, PureTemp LLC, Plymouth, MN, USA), PCR tubes, and aluminum heat sinks. PureTemp®-37 has a melting temperature of 37° C. The aluminum heat sinks allowed for increased thermal conductivity in the PCM. PCR tubes were placed inside of the center holes within the heat sinks. The PCM was melted within the mold using a hot plate before the heat sinks were placed. While it was still semi-soft, PCR tubes were placed within the heat sinks to shape the PCM. Power is provided by eight 1.2V nickel metal hydride (Ni-MH) AA batteries (EBL Mall, El Monte, CA, USA). A lid constructed from foam insulation is used during incubation. The structural components of the device and the assembly process can be seen in FIGS. 8-10.

Mock Reaction Temperature Profiles: For optimization and proof-of-concept experimentation, incubation using the POC device was performed with mock reaction volumes. Three PCR tubes filled with 50 μl of water were placed in the heat sinks in the device. Three k-type thermocouple probes were placed at the bottom of the outside of the PCR tubes to monitor the temperature throughout the experiment. Previous experimentation showed that the temperature difference between the inside and the outside of the PCR tube is negligible during incubation. During incubation, the device is powered for 15 minutes as the PCM approaches its melting point. After 15 minutes, the device is disconnected from the batteries, and the PCR tubes are inserted into the device. To monitor power usage, the voltage of the batteries was recorded before and after experimentation. All testing was performed at an ambient room temperature of around 23° C.

Mock Concurrent Reactions: To imitate POC conditions, mock reaction volumes (50 μl water) were incubated in the device 10 times consecutively. This was to show the capability of the device to be used many times in one setting. For this experiment, the device was heated for 15 minutes and then disconnected from the power source before 30 minutes of incubation. After one incubation was finished, PCR tubes were removed from the device, new tubes were inserted, and the device was again connected to the power source until the temperature of the device began to rise again. The time spent reheating alternated between 2 and 5 minutes. This was repeated 10 times. The temperature of all three tubes was recorded at each step, and the results were averaged. The voltage of the rechargeable batteries was recorded before and after the experiment was finished. All testing was performed at an ambient room temperature of around 23° C.

RPA Reactions: RPA was performed using TwistAmp® Liquid Basic kit from TwistDx (Maidenhead, UK). The DNA detection experiments were done using the 289 bp positive control provided in the kit. dNTPs were purchased from ThermoFisher Scientific Inc. (Waltham, MA, USA). RPA reactions were incubated for 30 minutes using the POC device. Samples incubated in a mini heat block VWR (Radnor, PA, USA) were used for controls and comparison. All RPA reactions were incubated for 30 minutes at approximately 37° C. Sample clean-up was done using the NucleoSpin Gel and PCR Clean-up kit (Macherey-Nagel GmbH & Co. KG, Düren, Germany). The amplification products were detected using the FlashGel™ system (Lonza, Basel, Switzerland). Molecular biology grade water was purchased from Quality Biological (Gaithersburg, MD, USA). A microcentrifuge, analog vortex mixer, and all other consumables were purchased from VWR.

### Results

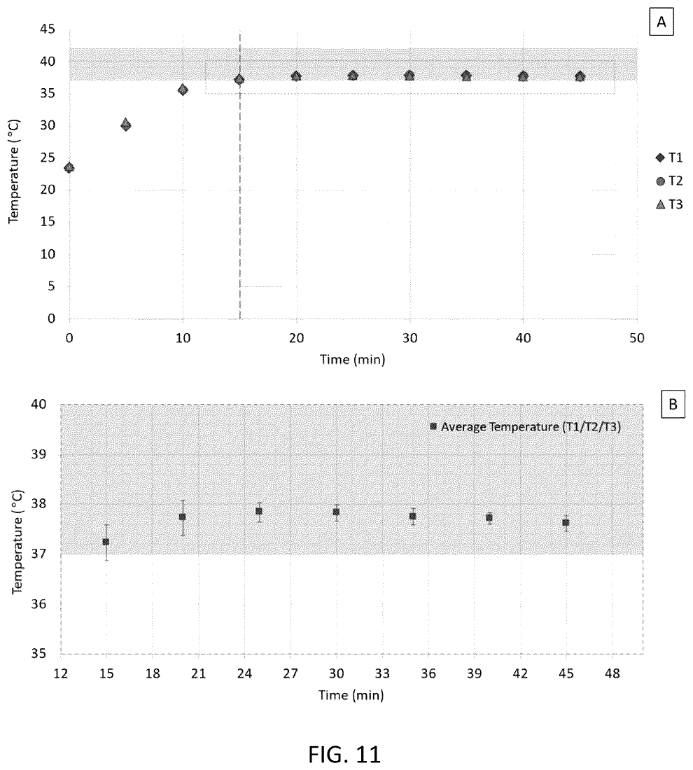

Temperature Profiles: While incubating mock reaction volumes in PCR tubes, the temperature profiles of the tubes were recorded. Consistently, all three tubes reached the target temperature range (37-42° C.) within 15 minutes and remained within that range for 30 minutes (FIG. 11). The average temperature of the tubes during the 30 minutes of incubation was 37.6° C. This temperature is sufficient to amplify live RPA reactions. The initial voltage of the fully charged Ni-MH batteries was about 11.50 V. After the fifteen minutes of heating, the voltage dropped about 0.50 V. This indicates that the batteries could be used for several experiments before needing to be recharged.

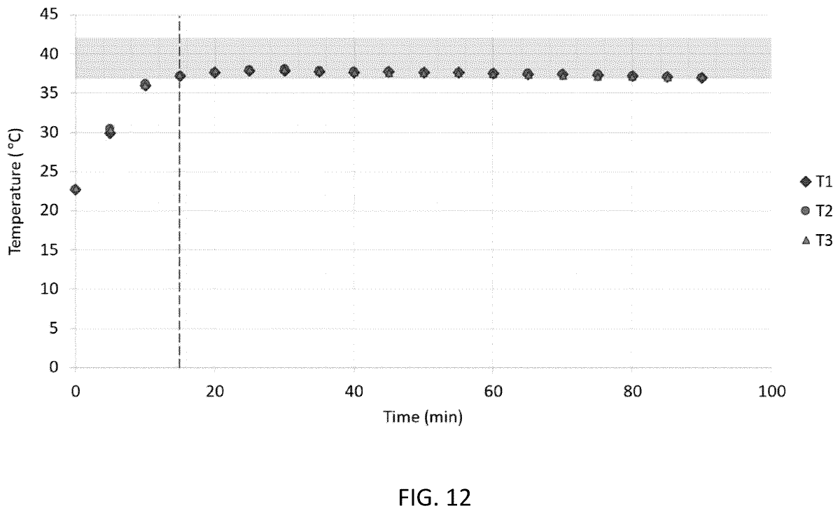

In the circumstances with a low pathogenic load in a sample, as might be the case in POC testing, an incubation period longer than the recommended 30 minutes might be desired. For this reason, the maximum time the device can hold the reaction in the target temperature range after 15 minutes of heating was recorded (FIG. 12). It was found that with 15 minutes of heating at room temperature, the device holds the tubes in the target temperature range for 75 minutes. This incubation time should be more than sufficient to incubate samples even with small pathogenic loads.

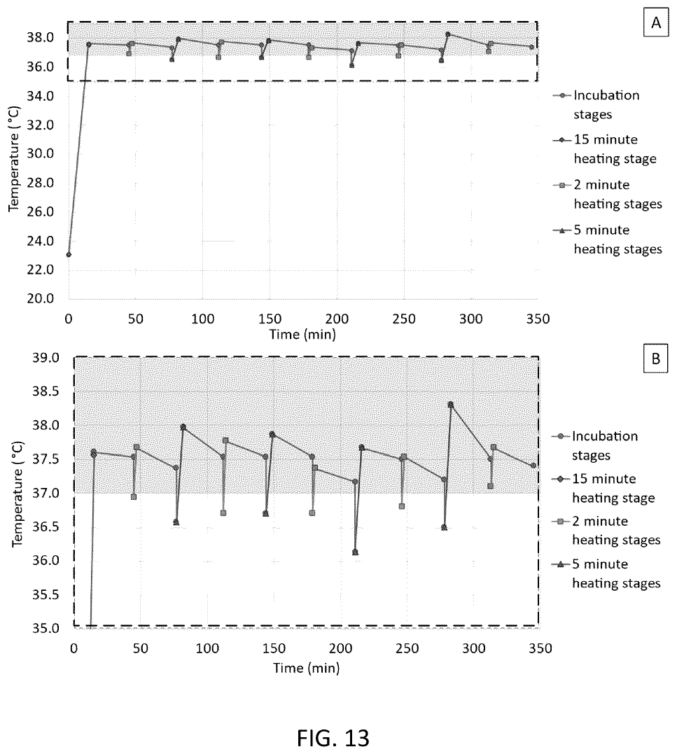

Concurrent Mock Reactions: In a point-of-care setting, it is possible that more than one round of testing must be performed using a single incubation device. Because PCMs have a high heat capacity, they are resilient to temperature fluctuations. Despite this, when the insulated POC device is opened to insert or remove samples, the exposure to the environment causes a small drop. in temperature. When using the device to incubate consecutive reactions, a small amount of reheating must be applied to the device between reactions. To explore this possibility, mock reactions were incubated 10 consecutive times. This corresponds to 30 reactions incubated. By starting with an initial 15-minute heating stage where the heater is was connected to the power source, then reconnecting the heat between incubation periods for 2 and 5 minutes, alternatively, all 10 incubations remained in the target temperature range (37-42° C.). As seen in FIG. 13, when opening the device, the temperature dropped as expected. However, the PCM stored enough thermal energy that bringing it back to the desired temperature range required small heating stages compared to the initial start-up. Alternating between longer and shorter heating periods was used to keep the required partially melted condition of the PCM stable: if the PCM solidifies, then the temperature will drop below the 37° C., and if it completely melts, then it will overheat, denaturing the enzymes used in RPA. Alternating between 2 and 5 minutes keeps the device warmer than consistent 3-minute heating steps and requires less overall heating time than consistent 4-minute heating steps.

The entire experiment took 345 minutes (5.75 hours) to complete. 300 minutes of this time was spent incubating reactions with the power disconnected. For the remaining 45 minutes, power was connected, and the device was being heated. This means that 10 incubation periods (30 reactions) can be completed with power only being provided for around 13% of the experiment. During this experiment, the voltage in the Ni-MH batteries started at 11.55 V. After the 45 minutes of use, the voltage had dropped to 10.09 V. It is reasonable to assume that several more consecutive incubations could have been performed using the intermittent heating method before the batteries fully lost charge.

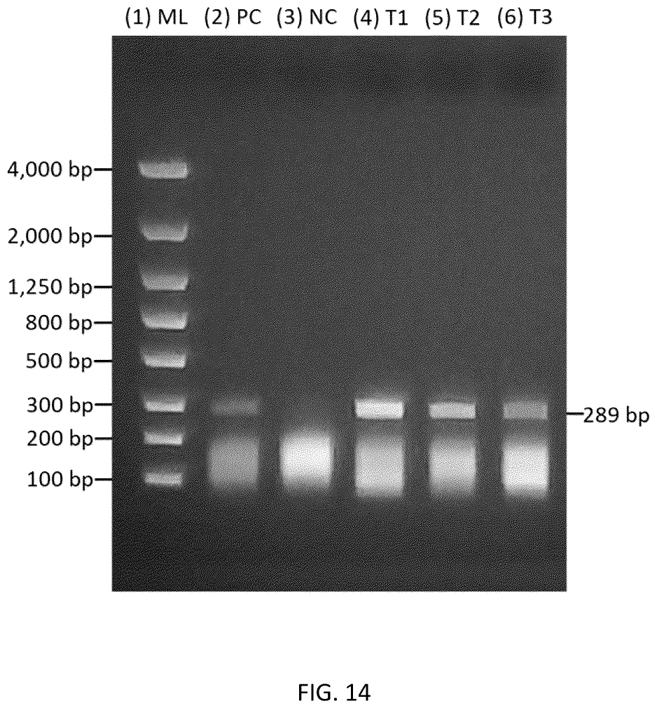

RPA Reactions: After the device was optimized, live RPA reactions were incubated in the device. Three RPA reactions were incubated in the device for 30 minutes. A positive and negative control were incubated in a heat block set to 37° C. Using the POC device, all three reactions were successfully amplified. This was confirmed using gel electrophoresis, as can be seen in FIG. 14. It is worth noting that gel electrophoresis is not an ideal detection method for POC settings, but was used in this case to confirm the success of the reaction; various POC detection methods exist that could be used with this device in future work. In lanes 4-6 of the gel a clear DNA band can be seen at the target molecular weight (289 bp). The bands for the samples incubated in the POC device are considerably stronger than those of the ones incubated in the heat block. This is likely because the heat block held the reactions steadily at 37° C., whereas the POC device reaches temperatures slightly over the 37° C., melting temperature of the PCM (about 37.6° C.) These results confirm that the POC device can be used to successfully incubate RPA reactions without any external power source or electronic temperature regulation.

### Discussion

Early experimentation with the POC device described in this work is shows promising results. Under standard ambient temperature conditions, the device is incredibly energy efficient, does not require the reset associated with the chemical heater, and utilizes very simple and user-friendly electronic components without needing a microcontroller. With minimal intermittent heating periods, the PCM in the device can be kept partially melted, and therefore, isothermal conditions, for long periods. This fact, combined with the fact that the device can incubate up to 3 samples at a time, indicates that it may be capable of performing consecutive, high-throughput POC testing. That said, more experimentation should be done to improve the device further.

As mentioned earlier, testing was performed under standard ambient temperature conditions. In a POC setting, a device needs to be functional in a wide range of conditions. It is likely that in colder conditions, longer heating periods, a higher voltage, or a heater with lower electrical resistance must be used in order to maintain the isothermal conditions required by RPA. All of these changes would impact the energy efficiency of the device. Alternatively, warmer ambient conditions might require shorter heating periods and lower voltages to prevent overheating. Future works will investigate this possibility. Regardless, experimentation showed that in some circumstances, the novel elements in this RPA POC device may offer advantages over existing devices using chemical and electric heating.

### Conclusions

The ability to rapidly diagnose infection diseases in POC settings is critical for overall global health. While existing POC tests are on the market, very few use NAATs, the most accurate and reliable was to diagnose infectious diseases. One of the biggest challenges when it comes to adapting NAATs for POC devices is temperature control. The advancements in isothermal NAATs such as RPA and LAMP make this challenge easier to overcome compared to the NAAT standard, PCR, but difficulties still exist. A portable heat source and temperature regulation system are required. The device presented in this work overcomes these challenges using a thin film polyimide heater and a PCM. The device follows the WHO ASSURED guidelines by building on existing strategies for NAAT POC testing with new innovations that mitigate previous disadvantages such as unreliable, hard to use heat sources and microcontrollers that add cost and complexity to the device. The device can simultaneously incubate three RPA reactions in a thermos cup using affordable, reusable parts and low energy consumption. It is completely portable and can easily incubate multiple reactions performed consecutively with very short intermittent heating periods.

### EXAMPLE 5

### A High-Throughput Diagnostic Point-Of-Care Device Capable of 8-12 Hour Continuous Use With Recombinase Polymerase Amplification

The Point-of-Care Incubator for Isothermal Nucleic Acid Amplification Test (PIINT) is a device that aims to meet the standards set by the WHO for point-of-care (POC) testing; these include the requirements of being equipment-free, user-friendly, robust, and affordable. The design of the PIINT regulates diagnostic reaction temperatures and minimizes temperature fluctuations in a manner that eliminates the need for electronic control circuitry. Recombinase polymerase amplification (RPA) is a nucleic acid amplification test that shows greater promise for POC testing. RPA operates at a temperature range of 37-42° C. In this work, the principal issues of temperature stability during incubation are addressed. With only a short initial heating period, the PIINT can maintain the target temperature range above the time required by the RPA reaction. Additionally, in a scenario mimicking on-site testing for about 12 hours, the PIINT, which holds three PCR tubes, was used to incubate 60 mock reaction volumes in 20 consecutive periods. Because of the PIINT's ability to mediate temperature fluctuations, the device could be opened to remove and replace samples, making successive incubations possible. No complications were present during the first 8 hours of the test. During the last 4 hours of testing, the device presented some unexpected behavior, likely due to repeated interrupted phase change, but still was able to incubate samples adequately. Due to the high thermal stability of the PIINT, the device was disconnected for 88% of the testing period. Only short intermittent heating periods were required to maintain isothermal conditions.

### Introduction

Infectious diseases kill millions of people each year and are considered one of the greatest WHAT faced by society. It has been estimated that up to half of the global population is at risk from infectious disease. They can be devastating on an individual level but also catastrophic on a communal and international scale. This risk has never been made more evident following the wake of the SARS-CoV-2 (COVID-19) pandemic, an illness that, according to the World Health Organization (WHO), may have caused as many as 3 million deaths in the year 2020 alone. Pandemics such as COVID-19 show the need for reliable, accurate, and timely testing. Access to testing was an important factor in which countries and regions faced the most devastating effects of COVID-19. Critically, new human-driven factors such as climate change, deforestation, large-scale livestock production, and antimicrobial resistance are predicted to accelerate and exasperate the threat of new and reemerging pathogens. Work must be done to increase global access to diagnostic testing. One method to improve testing in areas with limited or easily overfilled medical infrastructure is Point-Of-Care (POC) testing.

POC tests provide on-site results without the need to transport specimens to a laboratory. In low-resource settings, patients often must travel long distances to receive treatment. Even in middle-income countries, transporting samples from rural healthcare clinics to central laboratories prolongs the time required to get a diagnosis and introduces an additional chance for samples to be lost or damaged. POC testing removes the need for either the patient or the specimen to travel long distances for rapid diagnosis. However, because POC tests are performed outside of a clinical setting and often by individuals with little to no training, they must be simple, rugged, reliable, and capable of working in a variety of ambient conditions. The WHO has created a list of criteria for evaluating POC devices, the “ASSURED” standards. ASSURED stands for Affordable, Sensitive, Specific, User-Friendly, Rapid and Robust, Equipment-free, and Deliverable. Most POC diagnostic tests use lateral flow immunoassays (LFIA). Owing to the amount of sample required for an LFIA to avoid a false negative, there is a desire to make diagnostic methods such as nucleic acid amplification tests ASSURED.

The WHO has set a standard for POC tests that they must be no less than 80% sensitive to act as a replacement for clinical nucleic acid amplification tests or NAATs; rapid LFIA-based influenza tests have a sensitivity of about 50-70%, and LFIA-based COVID-19 tests have a sensitivity of about 70%. The gold standard for diagnosing infectious disease, the polymerase chain reaction (PCR), was the first NAAT created and is much more sensitive than any LFIA-based technology. However, PCR has many drawbacks as a POC method. PCR requires expensive and sophisticated equipment to perform. The sample must be placed in a thermocycler that rapidly takes the sample from 95° C. to 55° C. to 72° C. and repeat that process dozens of times. Therefore, PCR is very energy-extensive and is usually performed by trained experts in laboratories. However, since the invention of PCR in 1983, several isothermal NAATs have been developed with similar accuracy to PCR but a more simple mechanism. One such isothermal NAAT, recombinase polymerase amplification (RPA), will be the focus of this work.

Recombinase Polymerase Amplification (RPA) was developed by Piepenburg et al. in 2006 and has been commercialized by UK-based biotech company TwistD. RPA runs optimally in the temperature range of 37-42° C., making it much easier to set and control the reaction temperature compared to PCR. This work presents a device capable of incubating RPA reactions in the desired temperature range in a POC setting with batteries and a thin-film polyimide heater as the only electric components. This device is called the Point-of-Care Incubator for Isothermal Nucleic Acid Amplification Tests, or the “PIINT.” The PIINT uses a phase change material (PCM) to regulate temperature, making it very stable against temperature fluctuation without adding the complexity of a microcontroller. In this work, to mimic the kind of high-throughput testing that might be required during a pandemic or other outbreak, PIINT thermal characteristics were examined. We show that the design of PIINT is capable of continuous use for 12 hours, corresponding to 60 RPA reactions across 20 tests.

### Materials & Methods

Assembly of the PIINT: Several steps are taken to assemble the PIINT for use with RPA (FIGS. 8-10). The main components of the PIINT are housed in a steel vacuum-insulated vessel (Thermos LLC, Schaumburg, IL, USA). The vessel houses the internal components of the PIINT. A 12V/13 W 70 mm diameter thin film polyimide heater (ICstation, Guangdong, China) is placed in the bottom of the steel vessel. Twenty grams of the PCM PureTemp®-37 (PureTemp®- LLC, Plymouth, MN, USA) is placed in an aluminum cup and heated until liquid. Next, three aluminum heat sinks are placed into the PCM, and three empty 0.2 mL PCR tubes are placed into the heat sinks. When the PCM resolidifies, the PCR tubes are removed, and the aluminum cup is suspended over the heater inside the steel vessel. An internal foam insulation lid shaped to the contours of the aluminum cup is placed over the PCM. An external foam insulation lid is placed over the steel vessel. The heater is powered via 8 1.2V nickel metal hydride (Ni-MH) AA batteries (EBL Mall, El Monte, CA, USA) connected via a simple battery snap connector.

Mock RPA Reactions: To verify the capabilities of the PIINT to maintain the conditions necessary for RPA testing, experiments were conducted using mock reaction volumes consisting of 50 μL of water in 0.2 mL PCR tubes. Three sample tubes were added to the device; one tube was placed inside the center hole of each aluminum heat sink. To monitor the temperature during testing, a k-type thermocouple (Omega Engineering Inc., Norwalk, CT, USA) was placed at the bottom of the heat sinks, inside the PCM, and directly outside of the PCR tubes. Previous experimentation has demonstrated that the temperature difference between the outside wall of the PCR tube and the reaction volume inside the PCR tube is negligible. To begin the experiment, the heater is connected to the power source, eight fully charged rechargeable 1.2V Ni-MH AA batteries, for 15 minutes. At 15 minutes, the temperature of the PCM approaches the target temperature range (37-42° C.), and the heater is disconnected from the power source. After the PIINT is disconnected from the batteries, the three sample tubes are placed into the device and incubated for 30 minutes. The ambient temperature conditions in the laboratory were approximately 22° C. during testing.

12-Hour Consecutive Mock RPA Testing: To assess the viability of the PIINT in an all-day testing scenario, it was used to incubate mock reactions for 12 hours. The initial setup was identical to that used for a single reaction, as was described in the previous section. After the first 30 minutes of incubation, the PIINT was opened, and the first three sample tubes were removed. Three new sample tubes were then placed in the PIINT, the PIINT was closed, and the heater was reconnected to the batteries. The time that the PIINT remained open between tests was less than 20 seconds per testing period. Rather than the initial 15-minute heating period required to start the testing, much shorter interstitial heating periods were used to counter the heat loss caused by opening the PIINT; the interstitial heating periods ranged from 3-5 minutes between each test. After the PIINT reached the target temperature range once again (37-42° C.), the heater was disconnected from the batteries, and the new sample tubes were incubated for another 30 minutes. This was repeated for a total of 20 incubation periods, or 60 mock reactions incubated. At the 6-hour mark, a second set of 8 fully charged rechargeable 1.2V Ni-MH AA batteries replaced the first set to account for the voltage drop that occurred after the first 10 tests. The temperature was recorded at the beginning and end of each incubation period. The voltage of the batteries was recorded in the fully charged and used states. The ambient temperature conditions in the laboratory were approximately 23° C. during testing. This experiment was performed one time.

### Results & Discussion

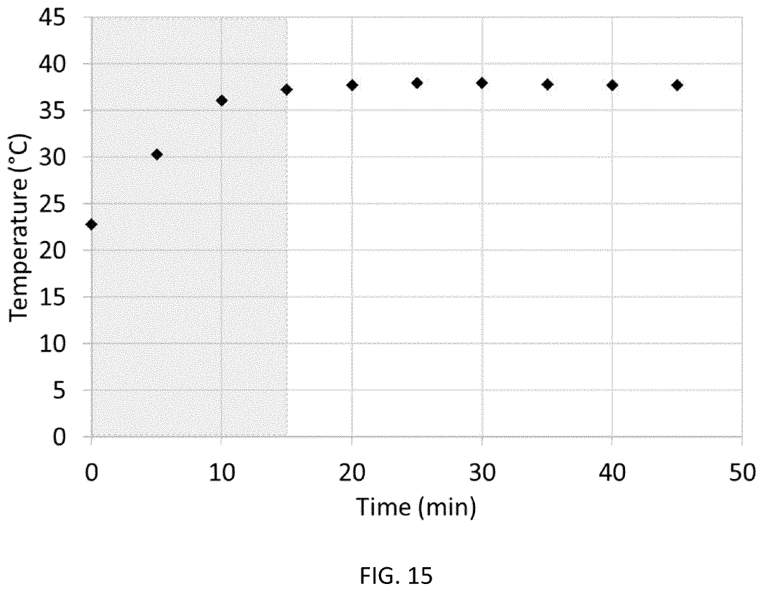

Temperature Profile for a Single RPA Reaction: It was found that in ambient temperature conditions, 15 minutes of heating was more than sufficient for samples in the PIINT to reach the target temperature range required for RPA (3742° C.). After the initial heating period, when the power was disconnected, the PIINT remained in the desired temperature range for the entire testing duration. Previous work has confirmed that the PIINT can amplify actual RPA reactions under these conditions. The temperature profile during testing can be seen in FIG. 15. After the 15-minute heating period, the voltage of the batteries dropped from 11.55 V to 10.48 V.

12-Hour Consecutive Testing: 20 consecutive 30-minute RPA incubations were performed using the PIINT. Three samples were incubated per test, so a total of 60 mock reactions were completed. This was achieved by initially heating the device for 15 minutes and then heating the device for short 3-5-minute bursts when PCR tubes were switched out. When the PIINT was opened to remove and replace sample tubes, the temperature in the PIINT would momentarily lower. However, the short heating periods were sufficient to bring the temperature back to the target temperature range (37-42° C.). At the beginning of testing, the first set of batteries had a voltage of 11.46 V. After 43 cumulative minutes of use, the voltage dropped to 9.86 V, and a new set of batteries was used going forward. The second set of batteries had an initial voltage of 11.48 V and dropped to 9.9 V after 41 minutes of cumulative use. In total, the experiment lasted 684 minutes/11.4 hours. The PIINT ran completely electricity-free for 88% of the time. For a single RPA test requiring 15-minute heating periods, the power is connected to the device for 33.3%. This demonstrates the power-efficient nature of the PIINT, especially when used for multiple consecutive tests.

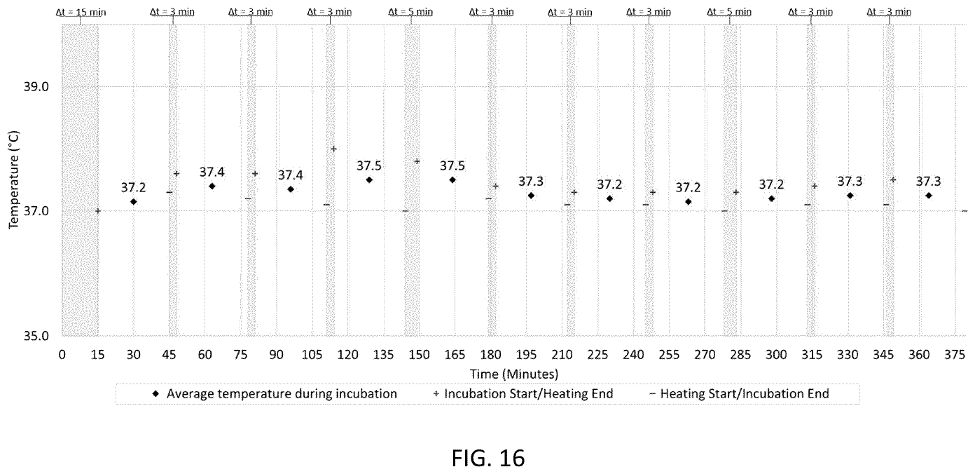

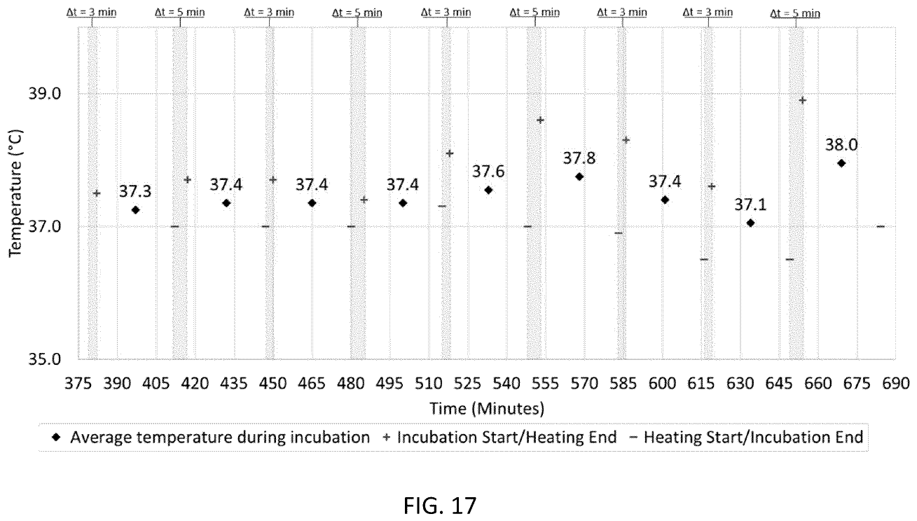

Towards the end of testing, beginning at approximately 8 hours into the testing period, more significant temperature fluctuations occurred between the beginning and end of testing. If the change in temperature fluctuations resulted in the system getting hotter overall, the results could perhaps be attributed to heat accumulation. While the PIINT was hotter on average past the 8-hour mark, both the lowest and highest temperatures recorded during each test moved further from the mean. This can be seen in Table 4. The standard deviation (SD) for the temperature during the last about 4 hours of testing of testing is more than double the SD for the first 8 hours: 0.68° C. compared to 0.27° C. The temperature profile for the first 8 hours of testing can be seen in FIG. 16. This corresponds to the first 11 incubation intervals, or the first 33 samples. FIG. 17 shows the temperature profile inside the PIINT for the remainder of the experiment. This corresponds to the last 9 incubations or the last 27 samples. As mentioned earlier, at this point in the testing period, the PIINT was performing in a way that was inconsistent with prior experimentation. Upon reviewing the literature, several factors may be influencing the results.

TABLE 4
Average temperature (T_{avg}) and SD across
different time intervals during tesing

| Time Interval | T_{avg} (° C.) | SD (° C.) |
|---|---|---|
| Start-8 hrs. | 37.3 | 0.27 |
| 8 hrs.-End | 37.4 | 0.68 |
| Start-End | 37.4 | 0.50 |

It has been well-documented that organic PCMs display a hysteresis effect between melting and refreezing. Typically, the temperature at which a PCM begins to resolidify after melting is lower than the melting temperature. Sometimes, the temperature at which solidifying occurs is even lower than expected; this is referred to as supercooling (also known as subcooling). Organic PCMs such as PureTemp®-37 tend to display very little supercooling under normal conditions. However, it has been shown that after multiple cycles of melting and solidifying, the hysteresis and degree of supercooling present can be affected. However, during individual incubations in the PIINT in which hours or days pass between tests, a change in performance has not been observed. Therefore, the issue of interrupted or “partial” phase change is more likely the problem.

Interrupted phase change occurs when a PCM is not allowed to fully transition from one phase to the other before heat flux is reversed. If there is one thing that researchers agree on regarding interrupted phase change, it is that the phenomenon is understudied and highly complex. Given that all POC devices that rely on PCM for temperature regulations utilize partial phase change by design, it should be of great interest to researchers in this field. When incubating a single reaction in the PIINT, the PCM undergoes interrupted melting; that is, the PCM is partially melted and then left to solidify again. In these circumstances, the interrupted phase change causes the PCM to begin to solidify at a higher temperature than expected. However, when doing consecutive incubations, both interrupted melting and interrupted solidification occurs. During interrupted solidification, the PCM will begin to liquefy at lower-than-expected temperatures. Also, the rate of heating and rate of cooling affects the melting/solidification hysteresis as well; in the PIINT, where heat is supplied via a heater and cooling occurs passively in an insulated vessel, the rates would be dramatically different. On top of these issues, supercooling becomes more apparent during interrupted phase change as well.

Overall, the nature of interrupted phase change is complex, understudied, and variable, but based on the results of the experiment and the literature on the topic, it seems like a very likely cause for the strange behavior observed during testing. Practically, this experiment shows that while the PIINT was able to incubate all mock RPA reactions in the ideal temperature range, more reliable results might be obtained by limiting consecutive testing periods to around 8 hours and by replacing the PCM in the PIINT on a semi-regular basis.

### Conclusions

The need for reliable and effective POC devices has never been greater. Accurately diagnosing infectious diseases is vital for individual and global health. In addition to following the ASSURED guidelines, to account for mass testing, such as what is required during a pandemic, a POC device also needs to be able to have a fast and easy reset between testing. Given that only the RPA samples needed to be replaced during consecutive testing and the energy-efficient nature of the device, this experiment showed that the PIINT is a promising option for meeting these demands. While some unfavorable behavior occurred towards the end of the experiment, perhaps due to repeated interrupted phase change, the average temperature of the PIINT never dropped below 37° C. In the future, shorter consecutive testing periods and more frequent replacement of the PDK The ability to handle 60 samples in one sitting without a significant reset period has not previously been demonstrated in existing works focusing on NAAT POC devices. The PIINT is entirely portable, user-friendly, requires minimum instrumentation, and can handle a high throughput of samples.

### EXAMPLE 6

### A Point-Of-Care Device for the Incubation of Loop-Mediated Isothermal Amplification: Lamp in the PIINT

To stop the spread of infectious diseases, rapid diagnosis is vital. Point-of-care (POC) testing is a crucial diagnostics tool; this is especially true in low-resource settings where medical infrastructure is limited. However, false negatives from POC testing are a considerable risk compared to standard clinical testing. This is because in clinical settings, a nucleic acid amplification test (NAAT), or, more specifically, the polymerase chain reaction (PCR) is the gold standard. PCR is extremely sensitive and specific compared to the average POC test. However, because PCR requires complex thermocycling, its use has generally been limited to clinics. As a result, there has been increased interest in the isothermal NAAT loop-mediated isothermal amplification (LAMP). LAMP is even more sensitive and specific than PCR and can be incubated in a single temperature range, removing the need for thermocycling. This makes it easier to adapt to a POC device. In this work, a POC device named the Point-of-Care Incubator for Isothermal Nucleic Acid Amplification Tests, or the “PIINT,” will be used to incubate mock reaction volumes in the LAMP temperature range. This serves as a proof-of-concept test towards the goal of incubating LAMP in a POC setting.

### Introduction

Since its development by Notomi et al in 2000, loop-mediated isothermal amplification (LAMP) has been studied as an exciting alternative to polymerase chain reaction (PCR) and a possible point-of-care (POC) tool. LAMP is among the oldest isothermal NAATs and is one of the only isothermal NAATs approved for use by the Food and Drug Administration. Because LAMP is an isothermal reaction, it is simpler to incubate than PCR. This makes it a more suitable candidate for POC devices. Additionally, LAMP is faster than PCR, has a higher yield rate, requires less pathogenic material, and is more sensitive and specific. LAMP typically is incubated at 63° C. but can perform optimally throughout the range of 60-65° C. POC devices utilizing LAMP must be able to maintain these conditions for the duration of the reaction.

When designing a POC test, the goal should be to make it as rugged, simple, and equipment-free as possible. The more complex the design is, the more opportunities for breaks and malfunctions are present. This makes maintaining strict isothermal conditions a challenge. To avoid the need for a microcontroller or other complicated components, phase-change materials (PCMs) are commonly used to remove excess heat during isothermal POC incubations. This is because when a material is changing phases, the temperature of the material will remain at the melting point until the material has entirely transitioned to the new phase. This fact is taken advantage of by the Point-of-Care Incubator for Isothermal Nucleic Acid Amplification Tests, or the PIINT. The PIINT is a POC device that utilizes a battery-powered heater and PCM to regulate temperature during isothermal NAATs. Previous work has shown that the PIINT can be used to incubate the NAAT recombinase polymerase amplification. In this work, it is modified and tested for use with LAMP.

### Materials & Methods

Construction of the PIINT for Use with LAMP: The main body of the PIINT is a vacuum-insulated steel vessel. A 12V thin-film polyimide heater is placed at the bottom of the vessel to provide energy to the reactions. Above the heater, inside the vessel, an aluminum cup holds 30 grams of the PCM Pure Temp®-63, three aluminum heat sinks, and 3 sample tubes. PureTemp®-63 has a melting point of 63° C. An internal foam insulated lid is placed inside the aluminum cup. An external foam-insulated lid then goes outside of the steel vessel. The heater is connected to rechargeable AA batteries via a snap connector. Two sets of batteries, 12V and 6V, are used.

The goal while optimizing the PIINT for LAMP was to ensure that the device reached the target temperature range (60-65° C.) within 30 minutes and was able to maintain that range for at least 60 minutes. To test this, mock reaction volumes were used in place of live reactions. 0.2 ml PCR tubes were filled with 50 μl of water; this corresponds to the normal volume of a LAMP reaction. During testing, the PCR tubes were placed inside the center of the heat sinks with the reaction volume submerged into the PCM. To monitor the temperature throughout the reaction, k-type thermocouple probes were placed at the bottom of the heat sink directly outside of the sample tube. During the 30-minute warm-up phase, the heater was connected to a 12V power source comprising of 8 rechargeable AA batteries. For the 60-minute incubation phase, the voltage was dropped to 6V. To monitor power usage, the voltage of the batteries was recorded before and after experimentation. Testing was performed in a room with an ambient temperature of around 23° C.

### Results & Discussion

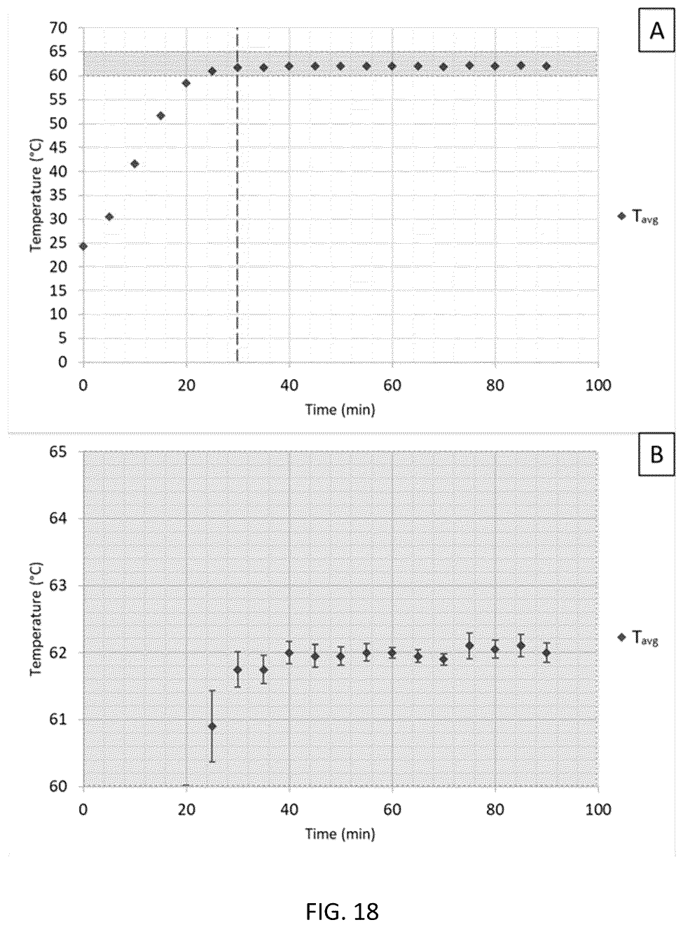

While incubating mock LAMP reactions, the PIINT reached the target temperature range in the desired time, and all three samples remained in the target temperature range for the duration of the incubation. The temperature profile during the experiment can be seen in FIG. 18. During testing, the average temperature during incubation was 61.9° C. FIG. 18. B includes the standard deviation for the temperatures recorded on different duplicates of the experiment. During incubation, the temperature inside the device never varied by more than 1° C. and never fell out of the desired range. According to the literature, this temperature should be sufficient to amplify LAMP reactions. During the warm-up period, the voltage in the batteries dropped from 11.46V to 10.18V. During incubation, the voltage dropped from 5.77V to 4.99V. At the end of each incubation, the PCM was partially melted. This is expected given the fact that completely melted PCM would cause the device to overheat. Overall, these results indicate that the PIINT could be used to incubate LAMP. Further testing is needed to confirm this possibility, but initial results are promising.

### Conclusions

Diagnostics using LAMP could aid in casing the global burden of disease. The potential is even greater in low-resource settings, where POC testing is vital. To make a viable POC device, temperature regulation must be reliable and non-instrument. The device presented in this work, the PIINT, takes advantage of the thermal properties of a PCM to meet this requirement. The heater and batteries are the most complex part of the design. These are simple components that are hard to break and easy to replace, making them ideal for POC settings. While future work needs to be done to confirm that the PIINT can incubate live LAMP reactions, early results presented in this work are encouraging.

### EXAMPLE 7

### Using SYBR Green 1 as a Visual Detection Method for RPA in the PIINT

Isothermal nucleic acid amplification tests (NAATs) are a promising technology that could advance the world of point-of-care (POC) diagnostics. Isothermal NAATs are sensitive, specific, and have simple incubation requirements, making them reliable for accurate diagnosis and eliminating the need for expensive equipment. Devices such as the one presented in this work are designed so that reactions can be performed in a fully portable and non-instrumented fashion. However, for a POC device to be complete, a method for determining the test results must be just as simple and rapid. For recombinase polymerase amplification (RPA), the use of the nucleic acid gel stain “SYBR Green I” (SG1) serves this purpose. By putting small volumes of SG1 into completed RPA reactions, a visual shift can be seen between positive and negative results. This phenomenon is even more apparent when viewed under UV light. This work investigates the viability of using SG1 as an RPA detection method in conjunction with a POC device, the “PIINT.” Additionally, the most optimal viewing conditions were determined.

### Introduction

To combat infectious diseases in low-resource settings, simple point-of-care (POC) diagnostic methods are vital. To create reliable POC devices, the World Health Organization has created a list of criteria for evaluating POC devices, the “ASSURED” standards; ASSURED stands for Affordable, Sensitive, Specific, User-Friendly, Rapid and Robust, Equipment-free, and Deliverable. Nucleic acid amplification tests (NAATs), such as the polymerase chain reaction (PCR), are amongst the most sensitive and specific methods for diagnosing infectious diseases. However, PCR requires intense thermocycling for the reaction to progress, making it difficult to perform without complex equipment. This limits its potential as a POC test. Fortunately, in the past few decades, many isothermal NAATs have been developed. One such NAAT, recombinase polymerase amplification (RPA), is especially promising as a POC method given its relatively low reaction temperature (37-42° C.) and short reaction time (10-30 minutes). Despite these advances, one of the biggest challenges in adapting NAATs into a POC platform is temperature regulation. A significant body of work exists developing devices to incubate NAATs in POC settings. The device used in this study, the Point-of-Care Incubator for Isothermal Nucleic Acid Amplification Tests, or, the “PIINT,” has been developed to this end.

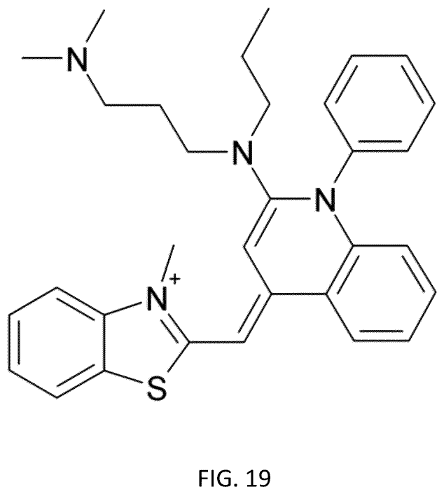

The PIINT is a portable device that can incubate RPA reactions in a POC setting, with the only electronic component being a battery-powered heater. However, up to this point, gel electrophoresis has been used to detect the RPA product. Just like temperature control, for a POC device to be fully functional, the results of the reaction must be detected in a way that complies with the ASSURED standards. Visual detection methods such as the use of SYBR Green I (SG1) are promising options. SG1 (FIG. 19) is a compound most often used as a nucleic acid gel stain. SG1 binds selectively to double-stranded DNA. When this occurs, the DNA-SG1 becomes fluorescent and causes a color shift that is visible to the naked eye. In RPA and most other NAATs, the target and the product is DNA. Therefore, by adding SG1 to completed RPA reactions, a visual shift will occur in those that are positive. Several authors have utilized this as an equipment-free detection method for RPA and other NAATs.

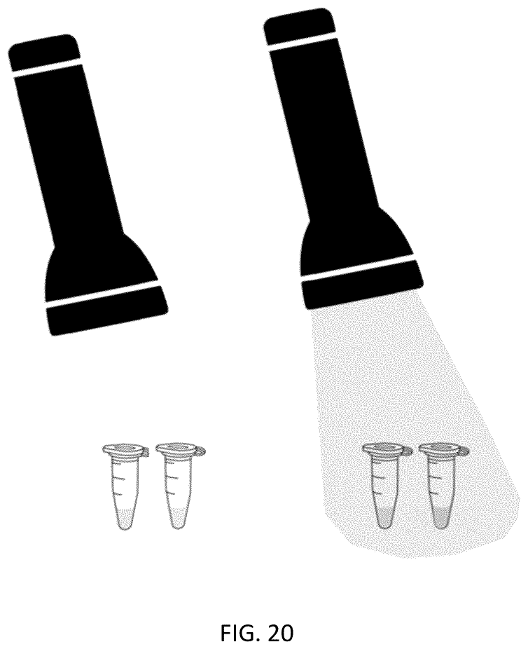

While the subtle visual change caused by SG1 may be a sufficient detection method on its own, a more obvious visual shift can be seen using UV light (see FIG. 20). SG1 has its highest fluorescent excitation peak between 500-550 nm wavelengths. However, it has a second excitation peak between 350-400 nm. This corresponds nicely with commercially available battery-powered UV torches; such torches often emit wavelengths at 365 nm and 395 nm. Therefore, by using SG1 and a simple UV torch, POC visual detection of RPA products is possible.

### Materials and Methods

RPA Reactions: RPA was performed according to manufacturer instructions using the TwistAmp® Liquid Basic kit from TwistDx (Maidenhead, UK). dNTPs were purchased from ThermoFisher Scientific Inc. (Waltham, MA, USA). RPA reactions were incubated for 30 minutes using the PIINT. Two kinds of target DNA were used to test SG1 detection; this included the positive control DNA provided in the TwistAmp® Liquid Basic kit and DNA from the bacterium Mycobacterium avium ssp. Paratuberculosis (MAP). Primers for MAP were purchased using Integrated DNA Technologies IDT Inc. (Coralville, IA, USA). For each reaction, a positive control (PC) and a negative control (NC) were performed.

Detection using SG1: After RPA reactions were completed, 3 μL of 375× SG1 in dimethyl sulfoxide for every 50 μL of the reaction volume. The SG1 was mixed into the reaction via pipetting. Next, pictures were taken of the reactions in natural lighting and then under UV lighting from a hand torch that emits both 365 and 395 nm wavelengths. Next, reactions were placed under two separate UV torches that emitted 365 nm and 396 nm wavelengths, respectively; this was to determine optimal viewing conditions.

### Results & Discussion

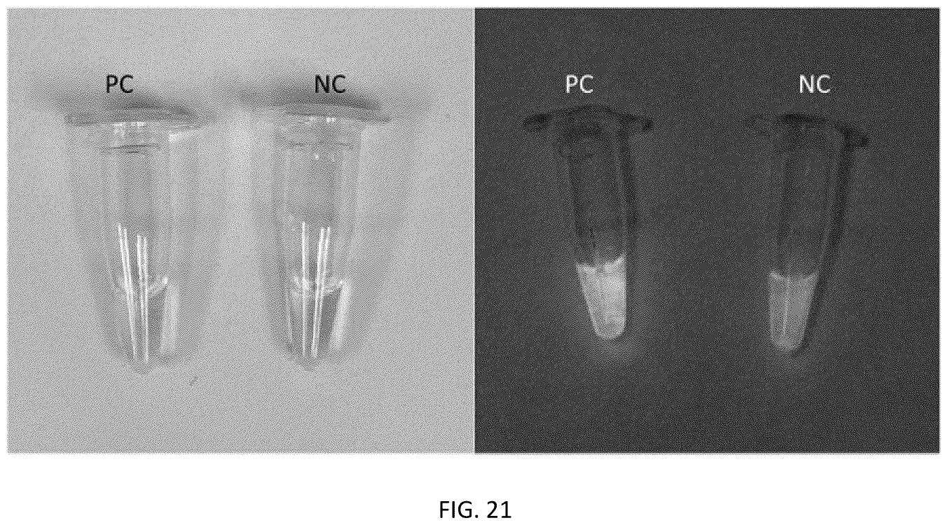

Using the PIINT as the incubator, RPA reactions were performed, and SG1 was added. When using the positive control provided by the RPA kit, a visual shift occurred, as can be seen in FIG. 21 (left). Under the 365/395 nm UV torch, the change was even more obvious.

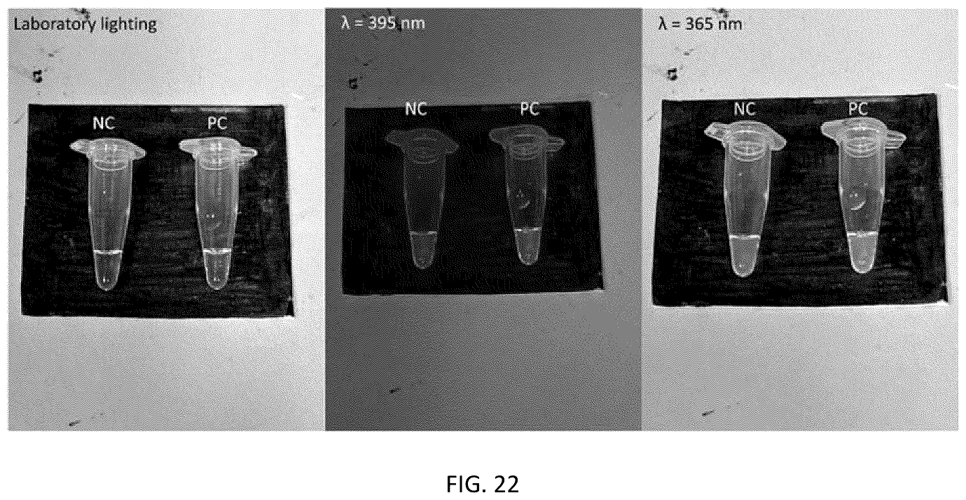

When performing RPA on the MAP target, the visual shift was not as apparent under laboratory lighting as with the TwistAmp® control target. This could be due to the presence of less DNA in the initial RPA reaction. Using a 395 nm UV torch did cause minor fluorescence to occur in the positive sample, but the blue cast made the results more challenging to read. The 395 UV torch gave the most obvious visual results. This can be seen in FIG. 22.

### Conclusions

When designing POC devices, all elements of the process must be considered. For isothermal NAATs such as RPA, both the temperature control and the detection method must be simple, portable, and equipment-free. Using the PIINT, a POC incubation method has been developed and verified. Now, in this work, a detection method has been successfully explored. Experiments have shown that SG1 is a viable visual detection method for POC applications. While sometimes the visual shift SG1 provides is obvious enough for naked eye detection, even results that do not produce an obvious color change in natural lighting can be confirmed via a UV torch. For this reason, SG1 is a promising option for NAAT POC diagnostics.

### EXAMPLE 8

### Point-Of-Care Detection of Johne'S Disease in Bovine Fecal Samples Using the PIINT

Point-of-care (POC) testing is vital for the effective treatment of infectious diseases, especially in low-resource settings. Although research into POC isothermal nucleic acid amplification testing (NAAT) is often focused on human diseases, animal and livestock diseases, e.g., Johne's disease, are ideal targets that would greatly benefit from POC testing in ways regarding both public/animal health and economics. Johne's disease (JD) is often diagnosed using the NAAT polymerase chain reaction (PCR) on fecal matter or through bacterial cultivation. Currently, these must be done in a laboratory; testing is generally only performed once a herd member shows clear symptoms. However, a significant percentage of the herd will usually be infected by the time an animal reaches this clinical stage. Therefore, regular testing using a POC device would significantly reduce the losses suffered by the herd due to JD. In this example, a simple and portable POC device using recombinase polymerase amplification (RPA), an isothermal NAAT, is explored. Using the Point-of-Care Incubator for Isothermal Nucleic Acid Amplification Tests, or the PIINT, fecal samples from cattle infected with JD were tested using RPA in the PIINT. This included using the PIINT to incubate the reactions and then using SYBR Green I as a POC visual detection method. With this method, JD was successfully detected in the fecal samples. This serves as a proof-of-concept trial to advance the feasibility of testing for JD outside of a traditional veterinary clinical setting.

### Introduction

In the wake of the devastating SARS-COV-2 pandemic, the threat posed by infectious disease has never been more apparent. However, while public consciousness has grown surrounding the dangers of the anthroponotic (human-to-human) spread of disease, around 60% of all known infectious diseases and 75% of emerging infectious diseases originate from zoonotic (animal-to-human) spread; this includes SARS-COV-2 and almost every other pandemic recorded in history. Additionally, zoonotic diseases always begin by existing exclusively within animal hosts. With an increase in factors such as climate change, land exploitation, globalization, and urbanization, contact between humans and animals carrying potential zoonotic diseases is higher than ever. As a result, beginning with an initiative put forward by the American Veterinary Medical Association, a concept called “One Health” has gained popularity. In basic terms, One Health is based on the idea that the health of humans, animals, and ecosystems are closely intertwined, and disease control requires multidisciplinary understanding and collaboration between experts in those fields. A quote by German physician Rudolf Virchow (1821-1902), known as “the father of modern pathology”, is often used in reference to One Health: “Between animal and human medicine, there is no dividing line, nor should there be”.

Johne's disease (JD) is a devasting livestock disease caused by the bacterium Mycobacterium avium ssp. Paratuberculosis (MAP). It affects ruminants and camelids and is most often found in dairy cattle. JD is a chronic wasting disease in which the absorption of food is hindered by intracellular MAP cells present in the intestinal wall. In cattle, symptoms can include weight loss, diarrhea, decreased milk production, and edema. If left to progress, an infected animal will die through cachexia (weight and muscle loss) and dehydration. Recently, JD has increasingly been investigated as a threat to human safety, some even going so far as to call it a “One Heath emergency”. While direct negative symptoms from MAP infection have not been seen in humans, MAP has been linked to a variety of human autoimmune responses. For decades, JD has been linked to Crohn's disease; this is due to both similarities in the immune response to both pathogens and the fact that MAP is found in Crohn's disease patients at seven times the volume than that of patients with any other bowel inflammation. While causation has never been proved, more and more research has recently been published, solidifying the link. Additionally, recent research has consistently found MAP to be a possible environmental trigger for the development of Type 1 diabetes.

The growing body of literature linking Johne's disease to human disorders is reason enough for concern, but even without that, mitigating JD is an urgent need for ranchers. The chronic, incurable disease can only be contained by culling an infected herd, and infected animals can remain in the subclinical (symptomless) state for years, all the while shedding MAP through fecal matter. Typically, animals are not tested until the clinical stage, and even then, symptoms can be hard to notice, especially in large herds. By the time an animal reaches the terminal stage, many other animals in the herd will already be in the clinical and subclinical stages; among dairy cattle, for every animal in the terminal stage, 15 to 25 other animals are likely infected. MAP is quite resilient; it can remain in water sources for nine months to two years, barn dust for several weeks, and bovine feces for 8-11 months. It is mainly contracted through a fecal-oral route, but it also can be transmitted through milk, intra-uterine dam-to-calf, the semen of infected bulls, and aerosolized particles. This puts neonatal calves and calves <6 months (therefore reliant on colostrum-containing milk) at the most significant risk of infection; heat treatments can be done on colostrum to reduce the number of MAP, but in order to completely kill the organism, temperatures would have to be so high that it would denature the vital antibodies in the colostrum itself. Beyond animal wellness, JD is also devastating financially; as of 2021, it is estimated that the United States, Germany, France, New Zealand, and Canada cumulatively lose $400-411 million USD annually due to JD (about $200 million from the United States alone). Ultimately, the livestock industry would greatly benefit from an affordable, sensitive point-of-care (POC) method of diagnosing JD.

All of this information paints a clear picture; testing herds for JD must be done often and reliably. Currently, testing is most often done on the fecal matter of suspected carriers through culture or polymerase chain reaction (PCR) to detect the bacteria or through ELISA to detect antibodies in blood serum or milk. However, cultures and ELISA tests have low sensitivity, especially in subclinical carriers, resulting in frequent false negatives. As a nucleic acid amplification test (NAAT), PCR is extremely sensitive but has been difficult to adapt as a POC method. PCR requires energy-extensive thermal cycling that is usually performed by trained professionals in laboratory settings with sophisticated equipment. However, alternative isothermal NAATs, such as recombinase polymerase amplification (RPA), are more straightforward to perform and, therefore, easier to adapt as a POC method. A POC device using RPA may serve as a tool for ranchers to detect Johne's disease early without having to wait on the time and expense of a lab.

This work explores the use of one such device, the PIINT, for the detection of JD in bovine fecal samples. The PIINT is portable, equipment-free, affordable, and has been shown to be able to successfully incubate RPA reactions. In these experiments, the PIINT is used to incubate samples during RPA using live samples infected with MAP bacteria rather than isolated DNA samples. This serves as a proof-of-concept trial for both the PIINT and for POC diagnostics of JD.

### Materials and Methods

Sample Collection & Verification: Fecal samples from cattle were collected and tested for JD by veterinary physicians at Logan Bethel Veterinary Services (Russellville, KY, USA). Over the course of 6 months, two cattle tested positive for JD. Both cases were diagnosed via fecal PCR. One fecal sample was primarily fluid with minimal solids, indicating a more advanced MAP infection. Once the samples were positively diagnosed for JD, they were stored in a −18° C. freezer for several months before testing with the PIINT was conducted. The samples from animals that had less advanced diarrhea and more advanced diarrhea are referred to as “Sample A” and “Sample B,” respectively.

DNA Extraction from Fecal Samples: To prepare the fecal samples for RPA, several cleaning steps were first performed for DNA extraction. First, a centrifugation method based on a modified version of a procedure described by Kameli et al. was used to concentrate the fecal samples. Approximately 3.5 mL of each frozen fecal sample was combined with 10 ml 1× PBS (pH 7.4). The tubes were then centrifuged at 3000 RPM for 15 minutes. Then, the PBS was discarded and replaced with a fresh solution, and the process was repeated. After concentrating the samples, they were further prepared using the QIAamp DNA Micro Kit (Qiagen N.V., Hilden, Germany) according to manufacturer recommendations.

PIINT Assembly: The assembly and function of the PIINT, as shown in FIGS. 8-10, have been described in detail in the previous sections. Briefly, the main internal components of the PIINT are housed inside a steel vacuum-insulated vessel (Thermos LLC, Schaumburg, IL, USA). A 12V/13 W 70 mm diameter thin film polyimide heater (ICstation, Guangdong, China) at the bottom of the vessel is held in place via small magnets. An aluminum cup placed above the heater inside of the PIINT holds 20 grams of the PCM Pure Temp®-37 (Pure Temp LLC, Plymouth, MN, USA), three aluminum heat sinks, and three 0.2 mL PCR tubes are placed into the heat sinks. An internal foam insulation lid is placed inside the PCM, and an external foam insulation lid is placed over the steel vessel. The heater is powered via 8 rechargeable 1.2V nickel metal hydride (Ni-MH) AA batteries (EBL Mall, El Monte, CA, USA).

RPA Primers & Analysis: For the RPA primers, two set of RxnReady Primer Pools (Integrated DNA Technologies IDT Inc., Coralville, IA, USA) were purchased. Preexisting literature was used to determine two sets of suitable primers for detecting MAP. Works by Zhao et al. and Hansen et al. both test multiple sequences for RPA for detecting MAP and report which sequences proved to be the most successful. For this study, the primers reported to have performed the best were used.

The primers arrived from the manufacturer as a dry pellet and were resuspended in dimethyl sulfoxide (DMSO) to a concentration of 10 μM. Both sets of primers were used during testing. After testing, the software OligoAnalyzer™ from IDT was used to further assess the viability of each set of primers and to troubleshoot any challenges that arose during experimentation.

RPA Reactions in the PIINT: RPA was performed using the TwistAmp® Liquid Basic kit from TwistDx (Maidenhead, UK). Reactions were performed with minor alterations according to the manual provided by TwistDx. Seven (7) μL of the primer mixes—3.5 μL of the forward and backward primers each—were used. Ten (10) μL of the cleaned stool sample elution was added to each reaction as the DNA template, bringing the total reaction volume to about 60 μL rather than the standard 50 μL. As a result, the volume of dNTPs (ThermoFisher Scientific Inc., Waltham, MA, USA) used in the reaction was adjusted according to manufacturer recommendations. To incubate the samples, the PIINT was first connected to the batteries and heated for 15 minutes until the device reached the target temperature of 37° C. After the device reached the desired temperature, the PIINT was disconnected from the power source, and the samples were placed into the center of the heat sinks and incubated for 30 minutes. The three tubes placed into the PIINT contained a negative control, Sample A and Sample B. This process was performed using both sets of primers and was repeated three times.

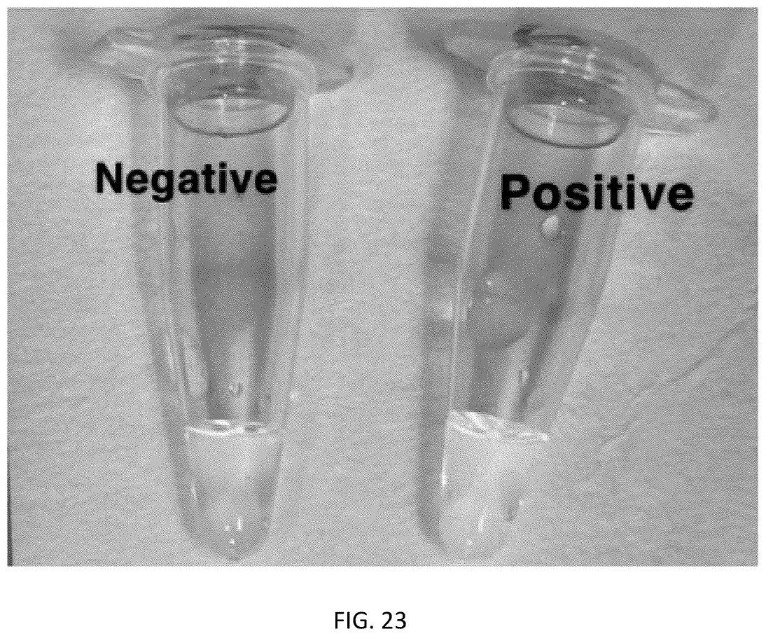

Visual detection using SYBR Green I: To visually detect the presence of RPA amplification products, SYBR Green I (SG1) was used. After performing RPA, 1.5 μL of 375× SG1 in DMSO was added per 30 μL of the reaction volume. The SG1 was then incorporated into the RPA mixture via pipette mixing. After the samples were mixed, a 365 nm ultraviolet (UV) flashlight was directed at the samples. Samples that are positive for JD will fluoresce with more intensity than the negative controls. An example of this can be seen in FIG. 23.

Because some fluorescence is still present in negative controls, it can be challenging to notice weakly positive results. For this reason, to determine how successfully users can determine positive results, six images containing randomly assorted arrangements of the negative control, Sample A and Sample B, were sent to 20 people. These people were asked to identify which two samples they believed were the positive results. Afterward, the percentage of times that the results were correctly interpreted was calculated.

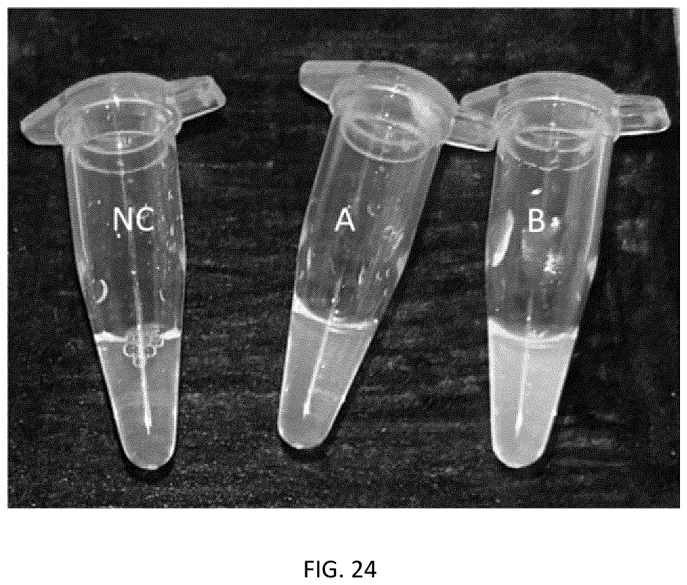

Detection of JD using RPA in the PIINT: Using the PIINT, RPA reactions were successfully incubated, and the results were analyzed. For reasons that are discussed in the following sections, the RPA reactions using the primers published by Hansen et al., were not included in this analysis. When performing RPA on Samples A and B and using SG1 as a detection method, it was consistent that Sample B fluoresced strongly, showing a clear positive result for JD, and Sample A fluoresced weakly, indicating a positive result for JD, but one that could possibly be mistaken for a false negative. This can be seen in FIG. 24. These results correlate with what is known about JD; namely, that cattle in more advanced stages of the disease have more liquid stool. Sample B having a stronger positive signal could imply that more DNA from the MAP bacteria was present compared to Sample A. This could indicate that RPA is most suitable for cattle in the more advanced stages of JD.

After 20 respondents were asked to identify the two positive results in 6 images showing the samples and the negative control, results were calculated; this can be seen in Table 5. Respondents were able to identify sample B as positive 100% of the time. When it came to sample A, respondents could identify it as positive 88% of the time.

TABLE 5
Percentage of respondents able to
identify samples positive for JD

| n* | sample | % of times identified as positive |
|---|---|---|
| 20 | A | 88% |
| 20 | B | 100% |

*n is the number of respondents.

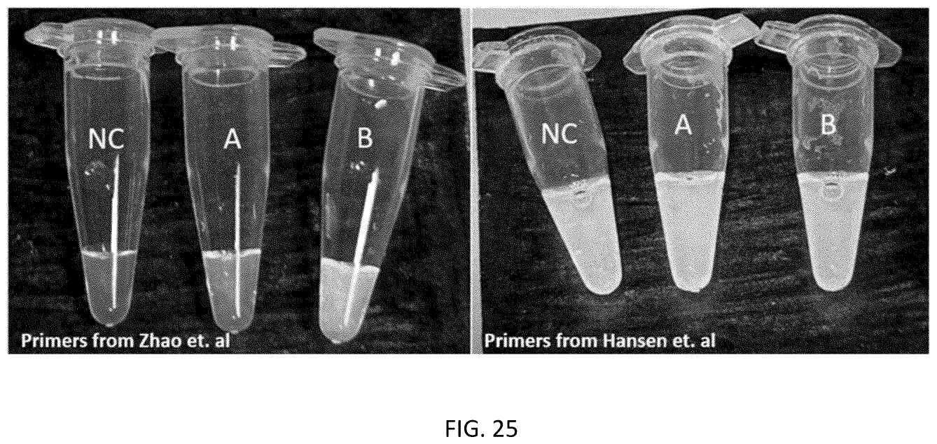

RPA Primers with SG1 detection: As mentioned previously, two sets of primers, one published by Zhao et al. and one published by Hansen et al., were used. When using SG1 as a visual detection method, the primers by Zhao et al. proved to be very successful. However, the primers by Hansen et al. were unsuitable in this instance. As can be seen in FIG. 25, RPA reactions that were amplified using the primers from Hansen et al. had significant fluoresces regardless of whether the vials contained Sample A, Sample B, or the negative control.

After repeated testing to ensure that the false positive was not a result of contamination or any other factors, the primer sequences were investigated. Using the software OligoAnalyzer™ from IDT, the likelihood of the primers forming “primer-dimers” was assessed. Primer-dimers occur when a primer binds to either another with the same sequence (a homo-dimer) or to a different primer (a hetero-dimer). In addition to using up reagents during NAATs, primer dimers cause off-target amplification. This is especially problematic when using SG1 as a detection method; SG1 binds to any double-stranded DNA, so non-specific amplification caused by primer dimers has a high likelihood of causing false positives.

A frequent method for determining the likelihood of dimer formation is to calculate the change in Gibbs free energy (AG) associated with primer hybridization. According to IDT, to minimize the risk of dimer formation, primers should have a AG greater than −9 kcal/mol. While a AG lower than −9 kcal/mol does not guarantee that primer-dimers will form, the lower AG it is, and the greater the number of configurations that would allow for primer hybridization, the more likely it is that primer-dimer-related issues will arise. The results of the OligoAnalyzer™ analysis can be seen in Table 6.

TABLE 6
OligoAnalyzer ™ analysis for the formation of primer-dimers

| | # of configurations with | Lowest ΔG |
|---|---|---|
| Sequence(s): | ΔG < −9 kcal/mol | (kcal/mol) |
| FP_H Self-Dimer | 2 | −9.75 |
| RP_H Self-Dimer | 4 | −12.96 |
| FP_H/RP_H Hetero- | 2 | −14.49 |
| FP_Z Self-Dimer | 1 | −13.19 |
| RP_Z Self-Dimer | 0 | −6.76 |
| FP_Z/RP_Z Hetero-Dimer | 0 | −6.78 |

For the primers published by Hansen et al., multiple configurations with ΔG lower than −9 kcal/mol exists for homo-dimers of both the forward and reverse primers and for hetero-dimers. This is in stark comparison to the primers published by Zhao et al., which only has one configuration at risk of developing a primer-dimer. Therefore, when using SG1 as a detection method, special care must be taken to ensure that the RPA primers will not cause a false-positive result.

### Conclusions

Rapid and reliable diagnosis of JD is of vital importance for animal welfare, the economy of the livestock industry, and possibly even human health. Regular POC testing is one method that can be implemented to advance this goal. NAATs are the best method to catch JD as early as possible. Because of the rapid reaction time and low temperature required by RPA, it is a promising method for POC diagnostics in general. Using the PIINT in combination with SG1, RPA can be performed and analyzed in a variety of settings without the need for lab equipment. However, non-specific amplification must be minimized to avoid falsely diagnosing a specimen. Overall, the results of this study show a glimpse into a possible future where ranchers such as those who took their cattle to the Logan Bethel Veterinary Clinic could have immediately tested for JD at the first sign of symptoms. The time it took for the veterinary clinic to get PCR results after sending fecal samples to a laboratory was one week. Diagnosing JD even a single week earlier could significantly slow the spread in a herd and prevent loss of life and livelihood for ranchers. The overall outcomes demonstrated in this work are promising for the eventual development of an RPA-based POC device for diagnosing JD.

In sum, this invention discloses, among other things, a portable, inexpensive incubator capable of maintaining isothermal conditions for POC NAATs. It offers a robust, minimally-instrumented, user-friendly way to diagnose infectious diseases in low-resource settings. This would allow for timely and accurate treatment for individuals in areas where diagnosing infectious diseases via traditional methods (i.e., in a clinical setting) is difficult. This would improve patient outcomes and could slow the spread of disease, benefiting both individual and global health.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### LIST OF REFERENCES

[1]. H. Hwang, B.-Y. Hwang and J. Bueno, Disease Markers, 2018, 2018, e8509127.
[2]. H. Ritchie and M. Roser, Our World in Data.
[3]. H. Chen, K. Liu, Z. Li and P. Wang, Clin Chim Acta, 2019, 493, 138-147.
[4]. The top 10 causes of death, https://www.who.int/news-room/fact-sheets/detail/the-top-10-causes-of-death, (accessed Oct. 5, 2021).
[5]. A. Suea-Ngam, L. Bezinge, B. Mateescu, P. D. Howes, A. J. deMello and D. A. Richards, ACS Sens., 2020, 5, 2701-2723.
[6]. WHO Coronavirus (COVID-19) Dashboard, https://covid19.who.int, (accessed Jul. 20, 2022).
[7]. P. Yager, G. J. Domingo and J. Gerdes, Annu Rev Biomed Eng, 2008, 10, 107-144.
[8]. L. Samuel, Clin Lab Med, 2020, 40, 483-494.
[9]. A. Karaivanov, S. E. Lu, H. Shigeoka, C. Chen and S. Pamplona, Journal of Health Economics, 2021, 78, 102475.
[10]. E. Rutayisire, G. Nkundimana, H. K. Mitonga, A. Boye and S. Nikwigize, International Journal of Infectious Diseases, 2020, 97, 267-269.
[11]. G. T. Hansen, Clinical Chemistry, 2020, 66, 124-137.
[12]. G. Niezen, P. Eslambolchilar and H. Thimbleby, BMJ Innovations, DOI:10.1136/bmjinnov-2015-000080.
[13]. G. Wu and M. H. Zaman, Bull World Health Organ, 2012, 90, 914-920.
[14]. P. K. Drain, E. P. Hyle, F. Noubary, K. A. Freedberg, D. Wilson, W. Bishai, W. Rodriguez and I. V. Bassett, Lancet Infect Dis, 2014, 14, 239-249.
[15]. M. G. Mauk, J. Song, C. Liu and H. H. Bau, Biosensors, 2018, 8, 17.
[16]. G. A. Obande and K. K. Banga Singh, Infect Drug Resist, 2020, 13, 455-483.
[17]. P. Maffert, S. Reverchon, W. Nasser, C. Rozand and H. Abaibou, Eur J Clin Microbiol Infect Dis, 2017, 36, 1717-1731.
[18]. P. Craw and W. Balachandran, Lab Chip, 2012, 12, 2469-2486.
[19]. V. Miralles, A. Huerre, F. Malloggi and M.-C. Jullien, Diagnostics (Basel), 2013, 3, 33-67.
[20]. J. Li, J. Macdonald and F. von Stetten, Analyst, 2018, 144, 31-67.
[21]. I. M. Lobato and C. K. O'Sullivan, TrAC Trends in Analytical Chemistry, 2018, 98, 19-35.
[22]. R. K. Daher, G. Stewart, M. Boissinot and M. G. Bergeron, Clinical Chemistry, 2016, 62, 947-958.
[23]. X. Qiu, S. Zhang, F. Xiang, D. Wu, M. Guo, S. Ge, K. Li, X. Ye, N. Xia and S. Qian, Sensors and Actuators B: Chemical, 2017, 243, 738-744.
[24]. U. F. Abdullahi, R. Naim, W. R. W. Taib, A. Saleh, A. Muazu, S. A. Baig and A. A. Baig, INDJST, 2015, 8, 1-12.
[25]. O. Piepenburg, C. H. Williams, D. L. Stemple and N. A. Armes, PLOS Biology, 2006, 4, e204.
[26]. J. R. Buser, S. Diesburg, J. Singleton, D. Guelig, J. D. Bishop, C. Zentner, R. Burton, P. LaBarre, P. Yager and B. H. Weigl, Lab Chip, 2015, 15, 4423-4432.
[27]. S. Huang, J. Do, M. Mahalanabis, A. Fan, L. Zhao, L. Jepeal, S. K. Singh and C. M. Klapperich, PLOS ONE, 2013, 8, e60059.
[28]. J. P. Goertz, K. M. Colvin, A. B. Lippe, J. L. Daristotle, P. Kofinas and I. M. White, ACS Appl. Mater. Interfaces, 2018, 10, 33043-33048.
[29]. S.-C. Liao, J. Peng, M. G. Mauk, S. Awasthi, J. Song, H. Friedman, H. H. Bau and C. Liu, Sensors and Actuators B: Chemical, 2016, 229, 232-238.
[30]. T. Pardy, I. Tulp, C. Kremer, T. Rang and R. Stewart, PLOS ONE, 2017, 12, e0189968.
[31]. C. Liu, M. G. Mauk, R. Hart, M. Bonizzoni, G. Yan and H. H. Bau, PLOS ONE, 2012, 7, e42222.
[32]. H.-W. Veltkamp, F. Akegawa Monteiro, R. Sanders, R. Wiegerink and J. Lötters, Micromachines (Basel), 2020, 11, E238.
[33]. J. Singleton, C. Zentner, J. Buser, P. Yager, P. LaBarre and B. H. Weigl, Proc SPIE, 2013, 8615, 86150R.
[34]. L. Schultz. University of Arkansas, 2021.

What is claimed is:

1. A point-of-care (POC) device for isothermal nucleic acid amplification tests (NAAT), comprising:
a housing;
a heater disposed in the housing;
at least one heat sink disposed in the housing in thermal communications with the heater, and configured to speed up heating and hold reaction tubes containing the NAAT being incubated; and
a phase change material (PCM) adapted for regulating temperature of the reactions.

2. The POC device of claim 1, wherein the housing is thermally insulated.

3. The POC device of claim 2, wherein the housing comprises both an external insulated lid and an internal insulated plug.

4. The POC device of claim 1, wherein the heater is placed in the bottom of the housing.

5. The POC device of claim 1, wherein the heater comprises a resistive heater of a thin-film polyimide heater.

6. The POC device of claim 1, wherein the heater comprises a positive temperature coefficient (PTC) heater, an electric or gas-powered flame, burning coal or embers, an external hot-plate or stove top, nichrome wire, and/or boiling water.

7. The POC device of claim 1, further comprising a power source for operably providing power to the heater.

8. The POC device of claim 7, wherein the power source comprises batteries.

9. The POC device of claim 8, wherein the batteries are rechargeable batteries.

10. The POC device of claim 1, further comprising a cup placed inside the housing in such a way that the cup is suspended over the heater without making direct contact with the heater.

11. The POC device of claim 10, wherein the cup is configured to hold the PCM, the reaction tubes, and the at least one heat sink.

12. The POC device of claim 11, wherein the cup is configured to hold the least one heat sink such that the at least one heat sink is in contact with the bottom of the cup and is partially submerged in the PCM.

13. The POC device of claim 12, wherein the at least one heat sink has center holes that are configured to fill with the PCM.

14. The POC device of claim 13, wherein the center holes are designed to hold the reaction tubes containing the NAAT so that the reaction volume is submerged in the PCM.

15. The POC device of claim 1, wherein the at least one heat sink is adapted to increase the thermal conductivity of the PCM allowing for rapid heating and electricity-free incubation.

16. The POC device of claim 12, further comprising an interior lid placed inside of the cup, and an insulated lid placed over the opening of the housing during incubation.

17. The POC device of claim 1, wherein the at least one heat sink is made of aluminum, copper, brass, or the like.

18. The POC device of claim 17, wherein the cup is made of a material that is same as or different from that of the at least one heat sink.

19. The POC device of claim 1, wherein the PCM is adapted for regulating the temperature to avoid the complexity of a microcontroller.

20. The POC device of claim 1, wherein the PCM is adapted such that the PCM has a melting temperature close to the target reaction temperature of a given NAAT to maintain an isothermal condition.

21. The POC device of claim 1, wherein the PCM is PureTemp®-37, which is a phase change material having a melting temperature of 37° C., for recombinase polymerase amplification (RPA).

22. The POC device of claim 1, wherein the PCM is PureTemp®-63, which is a phase change material having a melting temperature of 63° C., for loop-mediated isothermal amplification (LAMP).

23. The POC device of claim 1, having no electronic components.

24. The POC device of claim 1, being a point-of-care incubator for isothermal nucleic acid amplification tests (PIINT).

25. A method of point-of-care (POC) detection of Johne's disease (JD) in a subject, comprising:
preparing bio-samples of the subject;
incubating the bio-samples in the POC device of claim 1; and
visually detecting the JD from the incubated bio-samples.

26. The method of claim 25, wherein said preparing the bio-samples of the subject comprises
collecting the bio-samples from the subject;
cleaning and concentrating the bio-samples for DNA extraction;
determining suitable primers for detecting Mycobacterium avium ssp. Paratuberculosis (MAP); and
mixing the bio-samples and the primers for recombinase polymerase amplification (RPA) or loop-mediated isothermal amplification (LAMP) reactions.

27. The method of claim 25, wherein said incubating the bio-samples in the POC device comprises
heating the POC device until the POC device reaches a target temperature; and
placing the bio-samples into the center of the heat sinks and incubating them for a period of time to perform the RPA or LAMP reactions.

28. The method of claim 27, wherein the target temperature is about 37° C., and the period of time is about 30 minutes.

29. The method of claim 27, wherein the target temperature is about 63° C., and the period of time is about 60 minutes.

30. The method of claim 25, wherein said visually detecting the JD from the incubated bio-samples comprises
mixing an amount of SYBR Green I (SG1) with the incubated bio-samples to form a mixture thereof; and
illustrating the mixture with ultraviolet (UV) light at a wavelength of about 365 nm.

31. The method of claim 25, wherein the bio-samples that are positive for the JD fluoresce with more intensity than a negative control.
